(19) United States
(12) Patent Application Publication
(10) Pub. No.: US 2006/0074083 A1
(43) Pub. Date: Apr. 6, 2006

(54) Cyclic and acyclic propenones for treating CNS disorders

(75) Inventors: Ivars Kalvinsh, Salaspils (LV); Valerjans Kauss, Riga (LV); Dina Trifanova, Riga (LV); Ronalds Zemribo, Jurmala (LV); Wojciech Danysz, Nidderau (DE); Markus Henrich, Wetzlar (DE); Christopher Graham Raphael Parsons, Nidderau (DE); Tanja Weil, Heidelberg (DE)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: MERZ PHARMA GMBH & CO. KGAA, Frankfurt Am Main (DE)

(21) Appl. No.: 11/243,900

(22) Filed: Oct. 5, 2005

Related U.S. Application Data
(60) Provisional application No. 60/616,193, filed on Oct. 5, 2004.
(60) Provisional application No. 60/656,426, filed on Feb. 24, 2005.
(60) Provisional application No. 60/707,239, filed on Aug. 11, 2005.

(51) Int. Cl. A61K 31/5377; A61K 31/4741; A61K 31/405; A61K 31/353; A61K 31/343; C07D 491/02; C07D 413/02; C07D 405/02

(52) U.S. Cl. 514/232.5; 514/456; 514/469; 514/320; 514/422; 544/151; 544/153; 514/419; 546/196; 548/495; 549/467; 549/403; 514/302; 546/115

(57) ABSTRACT

The invention relates to novel cyclic and non-cyclic propenone derivatives as well as their pharmaceutically acceptable slats. The invention further relates to a process for the preparation of such compounds. The compounds of the invention are useful as medicaments.

### FIELD OF THE INVENTION

The present invention is concerned with novel metabotropic glutamate receptor (mGluR) modulators, methods for their synthesis and the treatment and/or prevention of neurological disorders by administration of such substances.

### BACKGROUND OF THE INVENTION

Neuronal stimuli are transmitted by the central nervous system (CNS) through the interaction of a neurotransmitter released by a neuron, which neurotransmitter has a specific effect on a neuroreceptor of another neuron.

L-glutamic acid is considered to be the major excitatory neurotransmitter in the mammalian CNS, consequently playing a critical role in a large number of physiological processes. Glutamate-dependent stimulus receptors are divided into two main groups. The first group comprises ligand-controlled ion channels whereas the second comprises metabotropic glutamate receptors (mGluR). Metabotropic glutamate receptors are a subfamily of G-protein-coupled receptors (GPCR). There is increasing evidence for a peripheral role of both ionotropic and metabotropic glutamate receptors outside the CNS e.g, in chronic pain states.

At present, eight different members of these mGluRs are known. On the basis of structural parameters such as sequence homology, the second messenger system utilized by these receptors and their different affinity to low-molecular weight compounds, these eight receptors can be divided into three groups: mGluR1 and mGluR5 belong to Group I which are positively coupled to phospholipase C and their activation leads to intracellular calcium-ion mobilization. Both mGluR2 and mGluR3 belong to Group II and mGluR4, mGluR6, mGluR7 and mGluR8 belong to Group III, both of which are negatively coupled to adenyl cyclase, i.e., their activation causes a reduction in second messenger cAMP and, as such, a dampening of neuronal activity.

Group I mGluR modulators have been shown to modulate the effects of the presynaptically released neurotransmitter glutamate via postsynaptic mechanisms. Moreover, as these modulators can be both positive and/or negative Group I mGluR modulators, such modulators may increase or inhibit the effects of these metabotropic receptors. Since a variety of pathophysiological processes and disease states affecting the CNS are thought to be related to abnormal glutamate neurotransmission and Group I mGluRs are shown to be expressed in several areas of the CNS, modulators of these receptors could be therapeutically beneficial in the treatment of CNS diseases.

Therefore, Group I mGluR modulators may be administered to provide neuroprotection in acute and chronic pathological conditions such as: AIDS-related dementia, Alzheimer's disease, Creutzfeld-Jakob's syndrome, bovine spongiform encephalopathy (BSE) or other prion related infections, diseases involving mitochondrial dysfunction, diseases involving β-amyloid and/or tauopathy such as Down's syndrome, hepatic encephalopathy, Huntington's disease, motor neuron diseases such as amyotrophic lateral sclerosis (ALS), multiple sclerosis (MS), olivoponto-cerebellar atrophy, post-operative cognitive deficit (POCD), Parkinson's disease, Parkinson's dementia, mild cognitive impairment, dementia pugilisitca, vascular and frontal lobe dementia, cognitive impairment, eye injuries or diseases (e.g. glaucoma, retinopathy, macular degeneration), head and spinal cord injuries/trauma, hypoglycaemia, hypoxia (e.g. perinatal), ischaemia (e.g. resulting from cardiac arrest, stroke, bypass operations or transplants), convulsions, glioma and other tumours, inner ear insult (e.g. in tinnitus, sound or drug-induced), L-dopa-induced and tardive dyskinesias.

Other indications in this context include a symptomatological effect on the following conditions: addiction (nicotine, alcohol, opiate, cocaine, amphetamine, obesity and others), amyotrophic lateral sclerosis (ALS), anxiety and panic disorders, attention deficit hyperactivity disorder (ADHD), restless leg syndrome, hyperactivity in children, autism, convulsions/epilepsy, dementia (e.g. in Alzheimer's disease, Korsakoff syndrome, vascular dementia, HIV infections), major depressive disorder or depression (including that resulting from Borna virus infection) and bipolar manic-depressive disorder, drug tolerance (e.g. to opioids), movement disorders, dystonia, dyskinesia (e.g. L-Dopa-induced, tardive dyskinesia or in Huntington's disease), fragile-X syndrome, Huntington's chorea, irritable bowel syndrome (IBS), migraine, multiple sclerosis, muscle spasms, pain (chronic and acute, e.g. inflammatory pain, neuropathic pain, allodynia, hyperalgesia, nociceptive pain), Parkinson's disease, post traumatic stress disorder, schizophrenia (positive and negative symptoms), spasticity, tinnitus, Tourette's syndrome, urinary incontinence and vomiting, pruritic conditions (e.g. pruritis), sleep disorders, micturition disorders, neuromuscular disorder in the lower urinary tract, gastroesophageal reflux disease (GERD), lower esophageal sphincter (LES) disease, functional gastrointestinal disorders, dyspepsia, regurgitation, respiratory tract infection, bulimia nervosa, chronic laryngitis, asthma (e.g. reflux-related asthma), lung disease, eating disorders, obesity and obesity-related disorders.

Yet further indications for Group I mGluR modulators include those indications wherein a particular condition does not necessarily exist but wherein a particular physiological parameter may be improved through administration of the instant compounds, for example cognitive enhancement.

Finally, propenones have been disclosed in the art. For example, Meng, et al. (US 2003/0236298) disclose specific 1,3-bis-aromatic-prop-2-en-1-ones for the treatment of VCAM-1 mediated disorders. Anderson, et al. (U.S. Pat. No. 6,864,264) disclose specific 1-adamantyl-3-aryl/heteroaryl-propenones for the treatment of proliferative disorders. Beckers, et al. disclose specific 2-acylindoles and their use as antitumor agents (WO 03/037861 and WO 01/082909). Hayakawa, et al. disclose specific 4-hydroxy-3-methyl-6-phenylbenzofuran-2-yl ketones and 4-hydroxy-3-methyl-6-phenylindol-2-yl ketones as antitumor agents. No metabotropic activity has been demonstrated for these compounds.

### THE PRESENT INVENTION

We have determined that certain cyclic and acyclic propenones are Group I mGluR modulators. Therefore, these substances may be therapeutically beneficial in the treatment of conditions which involve abnormal glutamate neurotransmission or in which modulation of Group I mGluR receptors results in therapeutic benefit. These substances are preferably administered in the form of a pharmaceutical composition, wherein they are present together with one or more pharmaceutically acceptable diluents, carriers, or excipients.

### OBJECTS OF THE INVENTION

It is an object of the present invention to provide novel pharmaceutical compounds which are cyclic and acyclic propenone Group I mGluR modulators and pharmaceutical compositions thereof. It is a further object of the invention to provide a novel method of treating, eliminating, alleviating, palliating, or ameliorating undesirable CNS disorders which involve abnormal glutamate neurotransmission by employing a compound of the invention or a pharmaceutical composition containing the same.

An additional object of the invention is the provision of a process for producing the cyclic and acyclic propenone active principles. Yet additional objects will become apparent hereinafter, and still further objects will be apparent to one skilled in the art.

### SUMMARY OF THE INVENTION

What we therefore believe to be comprised by our invention may be summarized inter alia in the following words:
A compound of formula I 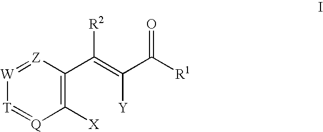
wherein
R¹ represents C₁₋₆alkyl, cycloC₃₋₁₂alkyl, C₂₋₆alkenyl, aryl, arylC₁₋₆alkyl, arylC₂₋₆alkenyl, heteroaryl, heteroarylC₁₋₆alkyl, arylC₃₋₆cycloalkyl, heteroarylC₂₋₆alkenyl, 2,3-dihydro-1H-indenyl, cycloC₃₋₁₂alkyl or cycloC₃₋₁₂alkylC₁₋₆alkyl, wherein the cycloC₃₋₁₂alkyl is optionally unsaturated and wherein one or more carbon atoms of the cycloC₃₋₁₂alkyl moiety may optionally be replaced by an oxygen atom or an NR⁷-moiety;
R² represents hydrogen or C₁₋₆alkyl;
X represents hydrogen, C₁₋₆alkyl, halogen, cyano, C₁₋₆alkoxy, nitro, or di-(C₁₋₆alkyl)amino (e.g. dimethylamino);
Y represents hydrogen, halogen, cyano, C₁₋₆alkyl, C₁₋₆alkoxy, hydroxyC₁₋₆alkyl, or di-C₁₋₆alkylaminoC₁₋₆alkyl; or
X and Y together may form a bivalent radical selected from OCR⁹R¹⁰, CH₂CR⁹R¹⁰, oxygen, CH₂, and N(R⁸);
Q represents nitrogen or R³—C;
T represents nitrogen or R⁴—C;
W represents nitrogen or R⁵—C;
Z represents nitrogen or R⁵—C;
   wherein
R³, R⁴, R⁵ and R⁶ each independently represents a hydrogen atom, a halogen atom, or a group selected from hydroxy, cyano, nitro, C₁₋₆alkyl, hydroxyC₁₋₆alkyl, C₁₋₆alkoxyC₁₋₆alkyl, aryl, arylC₁₋₆alkyl, heteroaryl, C₁₋₆alkoxy, cycloC₃₋₁₂alkoxy, arylC₁₋₁₆alkoxy, amino, C₁₋₆alkylamino, di-(C₁₋₆alkyl)amino, cycloC₃₋₁₂alkylamino, cycloC₃₋₁₂alkylC₁₋₁₆alkylamino, di-(C₁₋₆alkyl)aminoC₁₋₆alkyl, arylamino, arylC₁₋₁₆alkylamino, N-aryl-N-C₁₋₆alkylamino, C₁₋₆alkylcarbonylamino, N-C₁₋₆alkyl-N-C₁₋₆alkylcarbonylamino, pyrrolidino, piperidino, 4-C₁₋₆alkyl-piperazino, morpholino, hexamethyleneimino, pyrrolidinylC₁₋₆-alkyl, piperidinylC₁₋₆alkyl, morpholinylC₁₋₆alkyl, C₁₋₆alkylsulfonyl, C₁₋₆alkylsulfonylamino, C₁₋₆alkylsulfanyl, C₁₋₆alkylaminosulfonyl, and di-(C₁₋₆alkyl)aminosulfonyl;
R⁴ and R⁵ together may form a bivalent radical selected from —(CH₂)₃—, —(CH₂)₄—, —CH═CH—CH═CH—, —(CH₂)₃O—, —OCH₂O—, —O(CH₂)₂O—, and —O(CH₂)₃—;
R⁷ represents hydrogen, C₁₋₆alkyl, aryl, or cycloC₃₋₁₂alkylC₁₋₆alkyl;
R⁸ represents hydrogen, C₁₋₆alkyl or di-(C₁₋₆alkyl)aminocarbonyl;
R⁹ and R¹⁰ represent hydrogen or C₁₋₆alkyl;
and optical isomers, pharmaceutically acceptable salts, hydrates, solvates, and polymorphs thereof;
it being understood that:
aryl represents phenyl or naphthyl, or phenyl substituted by one or more substituents, which may be the same or different, selected from halogen, trifluoromethyl, trifluoromethoxy, C₁₋₆alkyl, C₂₋₆alkenyl, C₁₋₆alkoxy, amino, hydroxy, nitro, cyano, C₁₋₆alkoxycarbonyl, C₁₋₆alkylamino, di-(C₁₋₆alkyl)amino and C₁₋₆alkylenedioxy;
heteroaryl represents a (hetero)aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substitued by one or more substituents, which may be the same or different, selected from halogen, trifluoromethyl, C₁₋₆alkoxy, amino, hydroxy, nitro, cyano, C₁₋₆alkoxycarbonyl, C₁₋₆alkylamino, and di-(C₁₋₆alkyl)amino;
if Y represents hydrogen or C₁₋₆alkyl and R¹ represents aryl, then the ring formed by the substituents Q, T, W, and Z may not represent phenyl or substituted phenyl;
if R⁸ represents hydrogen, then R¹ may not represent C₁₋₆alkyl; phenyl or phenyl substituted by one or more groups selected from halogen, alkoxy, trifluoromethyl, alkyl, nitro, and amino; naphthyl; isoquinolinyl; 2-pyridyl; or 2-thienyl;
and the compound of formula I may not represent:
Cyclopropyl(5-methoxy-1H-2-indolyl)-1-methanone,
Cyclobutyl(5-methoxy-1H-2-indolyl)-1-methanone,
1-Adamantan-1-yl-3-quinolin-3-yl-propenone,
(6-Methoxy-2-benzofuran-2-yl)-(3-methoxyphenyl)-methanone,
1-Cyclopropyl-3-(3-methoxypheny)-propenone,
1-(3-Methoxyphenyl)-4,4-dimethyl-pent-1-en-3-one,
1-Adamantan-1-yl-3-(3,4,5-trimethoxyphenyl)-propenone,
1-Adamantan-1-yl-3-phenyl-propenone,
4-(3-oxo-3-(1-adamantyl)-prop-1-enyl)benzonitrile,
1-Adamantan-1-yl-3-(4-nitrophenyl)-propenone,
1-Adamantan-1-yl-3-(4-chlorophenyl)-propenone,
1-Adamantan-1-yl-3-(4-dimethylaminophenyl)-propenone,
1-Adamantan-1-yl-3-(4-isopropylphenyl)-propenone,
1-Adamantan-1-yl-3-(4-methoxyphenyl)-propenone,
1-Adamantan-1-yl-3-(4-fluorophenyl)-propenone,
1-Adamantan-1-yl-3-(2-bromophenyl)-propenone,
1-Adamantan-1-yl-3-(4-benzyloxyphenyl)-propenone,
1-Adamantan-1-yl-3-(4-biphenyl)-propenone,
1-Adamantan-1-yl-3-(4-ethylphenyl)-propenone,
1-Adamantan-1-yl-3-pyridin-2-yl-propenone,
1-Adamantan-1-yl-3-pyridin-3-yl-propenone,
1-Adamantan-1-yl-3-pyridin-4-yl-propenone,
1-Adamantan-1-yl-3-(6-methylpyridin-2-yl)-propenone,
1-Adamantan-1-yl-3-quinolin-4-yl-propenone,
1-Adamantan-1-yl-3-quinolin-2-yl-propenone, or
1-Adamantan-1-yl-3-thiophen-2-yl-propenone.

Compounds of formula I may be represented by the formula IA 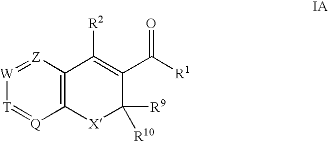
wherein
X′ represents oxygen or CH₂;
and optical isomers, pharmaceutically acceptable salts, hydrates, solvates, and polymorphs thereof.

Compounds of formula I may be represented by formula IB 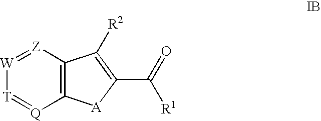
wherein
A represents oxygen, CH₂, or NR⁸;
and optical isomers, pharmaceutically acceptable salts, hydrates, solvates, and polymorphs thereof.

Compounds of formula I may be represented by formula IC 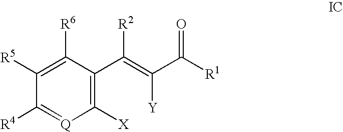
wherein
X represents hydrogen, C₁₋₆alkyl, halogen, cyano, C₁₋₆alkoxy, nitro, or di-(C₁₋₆alkyl)amino (e.g. dimethylamino); and
Y represents hydrogen, halogen, cyano, C₁₋₆alkyl, C₁₋₆alkoxy, hydroxyC₁₋₆alkyl, or di-C₁₋₆alkylaminoC₁₋₆alkyl;
and optical isomers, pharmaceutically acceptable salts, hydrates, solvates, and polymorphs thereof.

An additional embodiment of the present invention may be represented by the formula I′ 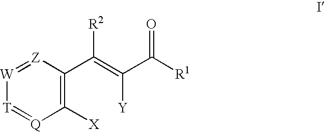
wherein
R¹ represents C₁₋₆alkyl, cycloC₃₋₁₂alkyl, C₂₋₆alkenyl, aryl, arylC₁₋₆alkyl, arylC₂₋₆alkenyl, heteroaryl, heteroarylC₁₋₆alkyl, arylC₃₋₆cycloalkyl, heteroarylC₂₋₆alkenyl, 2,3-dihydro-1H-indenyl, cycloC₃₋₁₂alkyl or cycloC₃₋₁₂alkylC₁₋₆alkyl, wherein the cycloC₃₋₁₂alkyl is optionally unsaturated and wherein one or more carbon atoms of the cycloC₃₋₁₂alkyl moiety may optionally be replaced by an oxygen atom or an NR⁷-moiety;
R² represents hydrogen or C₁₋₆alkyl;
X represents hydrogen, C₁₋₆alkyl, halogen, cyano, C₁₋₆alkoxy, nitro, or di-(C₁₋₆alkyl)amino;
Y represents hydrogen, halogen, cyano, C₁₋₆alkyl, C₁₋₆alkoxy, hydroxyC₁₋₆alkyl, or di-C₁₋₆alkylaminoC₁₋₆alkyl; or
X and Y together may form a bivalent radical selected from OCR⁹R¹⁰, CH₂CR⁹R¹⁰, and oxygen, CH₂, and N(R⁸);
Q represents nitrogen or R³—C;
T represents nitrogen or R⁴—C;
W represents nitrogen or R⁵—C;
Z represents nitrogen or R⁶—C;
wherein
R³, R⁴, R⁵ and R⁶ each independently represents a hydrogen atom, a halogen atom, or a group selected from hydroxy, cyano, nitro, C₁₋₆alkyl, hydroxyC₁₋₆alkyl, C₁₋₆alkoxyC₁₋₆alkyl, aryl, arylC₁₋₆alkyl, heteroaryl, C₁₋₆alkoxy, cycloC₃₋₁₂alkoxy, arylC₁₋₆alkoxy, amino, C₁₋₆alkylamino, di-(C₁₋₆alkyl)amino, cycloC₃₋₁₂alkylamino, cycloC₃₋₁₂alkylC₁₋₆alkylamino, di-(C₁₋₆alkyl)aminoC₁₋₆-alkyl, arylamino, arylC₁₋₆alkylamino, N-aryl-N-C₁₋₆alkylamino, C₁₋₆alkylcarbonylamino, N-C₁₋₆alkyl-N-C₁₋₆alkylcarbonylamino, pyrrolidino, piperidino, 4-C₁₋₆alkyl-piperazino, morpholino, hexamethyleneimino, pyrrolidinylC₁₋₆alkyl, piperidinylC₁₋₆alkyl, morpholinylC₁₋₆alkyl, C₁₋₆-alkylsulfonyl, C₁₋₆-alkylsulfonylamino, C₁₋₆alkylsulfanyl, C₁₋₆-alkylaminosulfonyl, and di-(C₁₋₆-alkyl)aminosulfonyl;
R⁴ and R⁵ together may form a bivalent radical selected from —(CH₂)₃—, —(CH₂)₄—, —CH═CH—CH═CH—, —(CH₂)₃O—, —OCH₂O—, —O(CH₂)₂O—, and —O(CH₂)₃—;
R⁷ represents hydrogen, C₁₋₆alkyl, aryl, or cycloC₃₋₁₂alkylC₁₋₆alkyl;
R⁸ represents hydrogen, C₁₋₆alkyl or di-(C₁₋₆-alkyl)aminocarbonyl;
R⁹ and R¹⁰ represent hydrogen or C₁₋₆-alkyl;
its optical isomers and pharmaceutically acceptable acid and base addition salts thereof;
it being understood that:
aryl represents phenyl or naphthyl, or phenyl substituted by one or more substituents, which may be the same or different, selected from halogen, trifluoromethyl, trifluoromethoxy, C₁₋₆alkyl, C₂₋₆alkenyl, C₁₋₆-alkoxy, amino, hydroxy, nitro, cyano, C₁₋₆alkoxycarbonyl, C₁₋₆alkylamino, di-(C₁₋₆alkyl)amino and C₁₋₆-alkylenedioxy;
heteroaryl represents a (hetero)aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substitued by one or more substituents, which may be the same or different, selected from halogen, trifluoromethyl, C₁₋₆alkoxy, amino, hydroxy, nitro, cyano, C₁₋₆alkoxycarbonyl, C₁₋₆alkylamino, and di-(C₁₋₆alkyl)amino;
if Y represents hydrogen or C₁₋₆alkyl and R¹ represents aryl, then the ring formed by the substituents Q, T, W, and Z may not represent phenyl or substituted phenyl;
if R⁸ represents hydrogen, then R¹ may not represent C₁₋₆alkyl; phenyl or phenyl substituted by one or more groups selected from halogen, alkoxy, trifluoromethyl, alkyl, nitro, and amino; naphthyl; isoquinolinyl; 2-pyridyl; or 2-thienyl;
if Y represents hydrogen, then R¹ may not represent adamantyl or adamantyl-C₁₋₆alkyl;
and the compound of formula I′ may not represent:
Cyclopropyl(5-methoxy-1H-2-indolyl)-1-methanone or
Cyclobutyl(5-methoxy-1H-2-indolyl)-1-methanone,
(6-Methoxy-2-benzofuran-2-yl)-(3-methoxyphenyl)-methanone,
1-Cyclopropyl-3-(3-methoxypheny)-propenone, or
1-(3-Methoxyphenyl)-4,4-dimethyl-pent-1-en-3-one.

Moreover, a method-of-treating a living animal, including a human for a condition associated with abnormal glutamate neurotransmission comprising the step of administering to the living animal an amount of an mGluR modulator selected from those of formula I 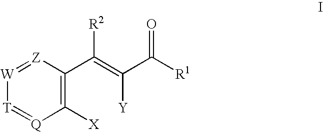
wherein
R¹ represents C₁₋₆alkyl, cycloC₃₋₁₂alkyl, C₂₋₆alkenyl, aryl, arylC₁₋₆alkyl, arylC₂₋₆alkenyl, heteroaryl, heteroarylC₁₋₆alkyl, arylC₃₋₆cycloalkyl, heteroarylC₂₋₆alkenyl, 2,3-dihydro-1H-indenyl, cycloC₃₋₁₂alkyl or cycloC₃₋₁₂alkylC₁₋₆-alkyl, wherein the cycloC₃₋₁₂alkyl is optionally unsaturated and wherein one or more carbon atoms of the cycloC₃₋₁₂alkyl moiety may optionally be replaced by an oxygen atom or an NR⁷-moiety;
R² represents hydrogen or C₁₋₆alkyl;
X represents hydrogen, C₁₋₆alkyl, halogen, cyano, C₁₋₆alkoxy, nitro, or di-(C₁₋₆alkyl)amino;
Y represents hydrogen, halogen, cyano, C₁₋₆alkyl, C₁₋₆alkoxy, hydroxyC₁₋₆alkyl, or di-C₁₋₆alkylaminoC₁₋₆alkyl; or
X and Y together may form a bivalent radical selected from OCR⁹R¹⁰, CH₂CR⁹R¹⁰, oxygen, CH₂, and N(R⁸);
Q represents nitrogen or R³—C;
T represents nitrogen or R⁴—C;
W represents nitrogen or R⁵—C;
Z represents nitrogen or R⁶—C;
wherein
R³, R⁴, R⁵ and R⁶ each independently represents a hydrogen atom, a halogen atom, or a group selected from hydroxy, cyano, nitro, C₁₋₆alkyl, hydroxyC₁₋₆alkyl, C₁₋₆alkoxyC₁₋₆alkyl, aryl, arylC₁₋₆alkyl, heteroaryl, C₁₋₆alkoxy, cycloC₃₋₁₂alkoxy, arylC₁₋₆alkoxy, amino, C₁₋₆alkylamino, di-(C₁₋₆alkyl)amino, cycloC₃₋₁₂alkylamino, cycloC₃₋₁₂alkylC₁₋₆alkylamino, di-(C₁₋₆alkyl)aminoC₁₋₆alkyl, arylamino, arylC₁₋₁₆alkylamino, N-aryl-N-C₁₋₁₆alkylamino, C₁₋₆alkylcarbonylamino, N-C₁₋₆alkyl-N-C₁₋₆alkylcarbonylamino, pyrrolidino, piperidino, 4-C₁₋₆alkyl-piperazino, morpholino, hexamethyleneimino, pyrrolidinylC₁₋₆alkyl, piperidinylC₁₋₆alkyl, morpholinylC₁₋₁₆alkyl, C₁₋₆alkylsulfonyl, C₁₋₆alkylsulfonylamino, C₁₋₆alkylsulfanyl, C₁₋₆alkylaminosulfonyl, and di-(C₁₋₆alkyl)aminosulfonyl;
R⁴ and R⁵ together may form a bivalent radical selected from —(CH₂)₃—, —(CH₂)₄—, —CH═CH—CH═CH—, —(CH₂)₃O—, —OCH₂O—, —O(CH₂)₂O—, and —O(CH₂)₃—;
R⁷ represents hydrogen, C₁₋₆alkyl, aryl, or cycloC₃₋₁₂alkylC₁₋₁₆alkyl;
R⁸ represents hydrogen, C₁₋₆alkyl or di-(C₁₋₆alkyl)aminocarbonyl;
R⁹ and R¹⁰ represent hydrogen or C₁₋₆alkyl;
it being understood that:
aryl represents phenyl or naphthyl, or phenyl substituted by one or more substituents, which may be the same or different, selected from halogen, trifluoromethyl, trifluoromethoxy, C₁₋₆alkyl, C₂₋₆alkenyl, C₁₋₆alkoxy, amino, hydroxy, nitro, cyano, C₁₋₆alkoxycarbonyl, C₁₋₆alkylamino, di-(C₁₋₆alkyl)amino and C₁₋₆alkylenedioxy;
heteroaryl represents a (hetero)aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substitued by one or more substituents, which may be the same or different, selected from halogen, trifluoromethyl, C₁₋₆alkoxy, amino, hydroxy, nitro, cyano, C₁₋₆alkoxycarbonyl,
C₁₋₆alkylamino, and di-(C₁₋₆-alkyl)amino;
and optical isomers, pharmaceutically acceptable salts, hydrates, solvates, and polymorphs thereof;
which is effective for alleviation of the condition.

Such a method-of-treating a living animal, including a human, for a condition associated with abnormal glutamate neurotransmission comprising the step of administering to the living animal an amount of an mGluR modulator selected from those of formula IA 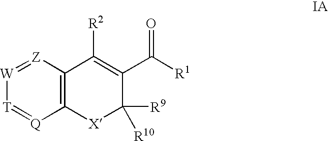
wherein
X′ represents oxygen or CH₂;
and optical isomers, pharmaceutically acceptable salts, hydrates, solvates, and polymorphs thereof;
which is effective for alleviation of the condition.

Such a method-of-treating a living animal, including a human, for a condition associated with abnormal glutamate neurotransmission comprising the step of administering to the living animal an amount of an mGluR modulator selected from those of formula IB 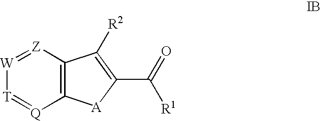
wherein
A represents oxygen, CH₂, or NR⁸;
and optical isomers, pharmaceutically acceptable salts, hydrates, solvates, and polymorphs thereof;
which is effective for alleviation of the condition.

Such a method-of-treating a living animal for a condition associated with abnormal glutamate neurotransmission comprising the step of administering to the living animal an amount of an mGluR modulator selected from those of formula IC 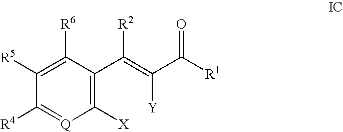
wherein
X represents hydrogen, C₁₋₆alkyl, halogen, cyano, C₁₋₆alkoxy, nitro, or di-(C₁₋₆-alkyl)amino; and
Y represents hydrogen, halogen, cyano, C₁₋₆alkyl, C₁₋₆-alkoxy, hydroxyC₁₋₆-alkyl, or di-C₁₋₆alkylaminoC₁₋₆-alkyl;
and optical isomers, pharmaceutically acceptable salts, hydrates, solvates, and polymorphs thereof;
which is effective for alleviation of the condition.

Such a method wherein the compound of formula I is administered in the form of a pharmaceutical composition thereof comprising the compound in combination with one or more pharmaceutically-acceptable diluents, excipients, or carriers.

Such a method wherein the condition associated with abnormal glutamate neurotransmission, or wherein modulation of mGluR receptors results in therapeutic benefit, is selected from the group consisting of AIDS-related dementia, Alzheimer's disease, Creutzfeld-Jakob's syndrome, bovine spongiform encephalopathy (BSE) or other prion related infections, diseases involving mitochondrial dysfunction, diseases involving β-amyloid and/or tauopathy such as Down's syndrome, hepatic encephalopathy, Huntington's disease, motor neuron diseases such as amyotrophic lateral sclerosis (ALS), multiple sclerosis (MS), olivoponto-cerebellar atrophy, post-operative cognitive deficit (POCD), Parkinson's disease, Parkinson's dementia, mild cognitive impairment, dementia pugilisitca, vascular and frontal lobe dementia, cognitive impairment, eye injuries or diseases (e.g. glaucoma, retinopathy, macular degeneration), head and spinal cord injuries/trauma, hypoglycaemia, hypoxia (e.g. perinatal), ischaemia (e.g. resulting from cardiac arrest, stroke, bypass operations or transplants), convulsions, glioma and other tumours, inner ear insult (e.g. in tinnitus, sound or drug-induced), L-dopa-induced and tardive dyskinesias.

Such a method wherein the condition associated with abnormal glutamate neurotransmission, or wherein modulation of mGluR receptors results in therapeutic benefit, is selected from the group consisting of addiction (nicotine, alcohol, opiate, cocaine, amphetamine, obesity and others), amyotrophic lateral sclerosis (ALS), anxiety and panic disorders, attention deficit hyperactivity disorder (ADHD), restless leg syndrome, hyperactivity in children, autism, convulsions/epilepsy, dementia (e.g. in Alzheimer's disease, Korsakoff syndrome, vascular dementia, HIV infections), major depressive disorder or depression (including that resulting from Borna virus infection) and bipolar manic-depressive disorder, drug tolerance (e.g. to opioids), movement disorders, dystonia, dyskinesia (e.g. L-Dopa-induced, tardive dyskinesia or in Huntington's disease), fragile-X syndrome, Huntington's chorea, irritable bowel syndrome (IBS), migraine, multiple sclerosis, muscle spasms, pain (chronic and acute, e.g. inflammatory pain, neuropathic pain, allodynia, hyperalgesia, nociceptive pain), Parkinson's disease, post traumatic stress disorder, schizophrenia (positive and negative symptoms), spasticity, tinnitus, Tourette's syndrome, urinary incontinence and vomiting, pruritic conditions (e.g. pruritis), sleep disorders, micturition disorders, neuromuscular disorder in the lower urinary tract, gastroesophageal reflux disease (GERD), lower esophageal sphincter (LES) disease, functional gastrointestinal disorders, dyspepsia, regurgitation, respiratory tract infection, bulimia nervosa, chronic laryngitis, asthma (e.g. reflux-related asthma), lung disease, eating disorders, obesity and obesity-related disorders.

Such a method wherein the condition associated with abnormal glutamate neurotransmission, or wherein modulation of mGluR receptors results in therapeutic benefit, is selected from indications wherein a particular condition does not necessarily exist but wherein a particular physiological parameter may be improved through administration of the instant compounds, including cognitive enhancement.

Further, a pharmaceutical composition comprising, together with one or more pharmaceutically acceptable excipients or vehicles, a compound selected from those of formula I 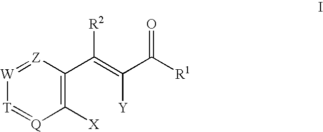
wherein
R¹ represents C₁₋₆alkyl, cycloC₃₋₁₂alkyl, C₂₋₆alkenyl, aryl, arylC₁₋₆alkyl, arylC₂₋₆alkenyl, heteroaryl, heteroarylC₁₋₆alkyl, arylC₃₋₆cycloalkyl, heteroarylC₂₋₆alkenyl, 2,3-dihydro-1H-indenyl, cycloC₃₋₁₂alkyl or cycloC₃₋₁₂alkylC₁₋₆alkyl, wherein the cycloC₃₋₁₂alkyl is optionally unsaturated and wherein one or more carbon atoms of the cycloC₃₋₁₂alkyl moiety may optionally be replaced by an oxygen atom or an NR⁷-moiety;
R² represents hydrogen or C₁₋₆alkyl;
X represents hydrogen, C₁₋₆alkyl, halogen, cyano, C₁₋₆alkoxy, nitro, or di-(C₁₋₆alkyl)amino;
Y represents hydrogen, halogen, cyano, C₁₋₆alkyl, C₁₋₆alkoxy, hydroxyC₁₋₆alkyl, or di-C₁₋₆alkylaminoC₁₋₆alkyl; or
X and Y together may form a bivalent radical selected from OCR⁹R¹⁰, CH₂CR⁹R¹⁰, oxygen, CH₂, and N(R⁸);
Q represents nitrogen or R³—C;
T represents nitrogen or R⁴—C;
W represents nitrogen or R⁵—C;
Z represents nitrogen or R⁶—C;
wherein
R³, R⁴, R⁵ and R⁶ each independently represents a hydrogen atom, a halogen atom, or a group selected from hydroxy, cyano, nitro, C₁₋₆alkyl, hydroxyC₁₋₆alkyl, C₁₋₆alkoxyC₁₋₆alkyl, aryl, arylC₁₋₆alkyl, heteroaryl, C₁₋₆alkoxy, cycloC₃₋₁₂alkoxy, arylC₁₋₆alkoxy, amino, C₁₋₆alkylamino, di-(C₁₋₆alkyl)amino, cycloC₃₋₁₂alkylamino, cycloC₃₋₁₂alkylC₁₋₆alkylamino, di-(C₁₋₆alkyl)aminoC₁₋₆alkyl, arylamino, arylC₁₋₆alkylamino, N-aryl-N—C₁₋₆alkylamino, C₁₋₆alkylcarbonylamino, N—C₁₋₆alkyl-N—C₁₋₆alkylcarbonylamino, pyrrolidino, piperidino, 4-C₁₋₆alkyl-piperazino, morpholino, hexamethyleneimino; pyrrolidinylC₁₋₆alkyl, piperidinylC₁₋₆alkyl, morpholinylC₁₋₆alkyl, C₁₋₆alkylsulfonyl, C₁₋₆alkylsulfonylamino, C₁₋₆alkylsulfanyl, C₁₋₆alkylaminosulfonyl, and di-(C₁₋₆alkyl)aminosulfonyl;
R⁴ and R⁵ together may form a bivalent radical selected from —(CH₂)₃—, —(CH₂)₄—, —CH═CH—CH═CH—, —(CH₂)₃O—, —OCH₂O—, —O(CH₂)₂O—, and —O(CH₂)₃—;
R⁷ represents hydrogen, C₁₋₆alkyl, aryl, or cycloC₃₋₁₂alkylC₁₋₆alkyl;
R⁸ represents hydrogen, C₁₋₆alkyl or di-(C₁₋₆alkyl)aminocarbonyl;
R⁹ and R¹⁰ represent hydrogen or C₁₋₆alkyl;
and optical isomers, pharmaceutically acceptable salts, hydrates, solvates, and polymorphs thereof;
it being understood that:
aryl represents phenyl or naphthyl, or phenyl substituted by one or more substituents, which may be the same or different, selected from halogen, trifluoromethyl, trifluoromethoxy, C₁₋₆alkyl, C₂₋₆alkenyl, C₁₋₆alkoxy, amino, hydroxy, nitro, cyano, C₁₋₆alkoxycarbonyl, C₁₋₆alkylamino, di-(C₁₋₆alkyl)amino and C₁₋₆alkylenedioxy; heteroaryl represents a (hetero)aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substitued by one or more substituents, which may be the same or different, selected from halogen, trifluoromethyl, C₁₋₆-alkoxy, amino, hydroxy, nitro, cyano, C₁₋₆-alkoxycarbonyl, C₁₋₆alkylamino, and di-(C₁₋₆-alkyl)amino;
if Y represents hydrogen or C₁₋₆-alkyl and R¹ represents aryl, then the ring formed by the substituents Q, T, W, and Z may not represent phenyl or substituted phenyl;
if R⁸ is hydrogen, then R¹ may not represent C₁₋₆alkyl; phenyl or phenyl substituted by one or more groups selected from halogen, alkoxy, trifluoromethyl, alkyl, nitro, and amino; naphthyl; isoquinolinyl; 2-pyridyl; or 2-thienyl;
and the compound of formula I may not represent:
Cyclopropyl(5-methoxy-1H-2-indolyl)-1-methanone,
Cyclobutyl(5-methoxy-1H-2-indolyl)-1-methanone,
1-Adamantan-1-yl-3-quinolin-3-yl-propenone,
(6-Methoxy-2-benzofuran-2-yl)-(3-methoxyphenyl)-methanone,
1-Cyclopropyl-3-(3-methoxypheny)-propenone,
1-(3-Methoxyphenyl)-4,4-dimethyl-pent-1-en-3-one,
1-Adamantan-1-yl-3-(3,4,5-trimethoxyphenyl)-propenone,
1-Adamantan-1-yl-3-phenyl-propenone,
4-(3-oxo-3-(1-adamantyl)-prop-1-enyl)benzonitrile,
1-Adamantan-1-yl-3-(4-nitrophenyl)-propenone,
1-Adamantan-1-yl-3-(4-chlorophenyl)-propenone,
1-Adamantan-1-yl-3-(4-dimethylaminophenyl)-propenone,
1-Adamantan-1-yl-3-(4-isopropylphenyl)-propenone,
1-Adamantan-1-yl-3-(4-methoxyphenyl)-propenone,
1-Adamantan-1-yl-3-(4-fluorophenyl)-propenone,
1-Adamantan-1-yl-3-(2-bromophenyl)-propenone,
1-Adamantan-1-yl-3-(4-benzyloxyphenyl)-propenone,
1-Adamantan-1-yl-3-(4-biphenyl)-propenone,
1-Adamantan-1-yl-3-(4-ethylphenyl)-propenone,
1-Adamantan-1-yl-3-pyridin-2-yl-propenone,
1-Adamantan-1-yl-3-pyridin-3-yl-propenone,
1-Adamantan-1-yl-3-pyridin-4-yl-propenone,
1-Adamantan-1-yl-3-(6-methylpyridin-2-yl)-propenone,
1-Adamantan-1-yl-3-quinolin-4-yl-propenone,
1-Adamantan-1-yl-3-quinolin-2-yl-propenone, or
1-Adamantan-1-yl-3-thiophen-2-yl-propenone.

Such a pharmaceutical composition comprising, together with one or more pharmaceutically acceptable excipients or vehicles, a compound selected from those of formula IA 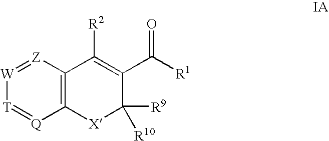
wherein
X′ represents oxygen or CH₂;
and optical isomers, pharmaceutically acceptable salts, hydrates, solvates, and polymorphs thereof;

Such a pharmaceutical composition comprising, together with one or more pharmaceutically acceptable excipients or vehicles, a compound selected from those of formula IB 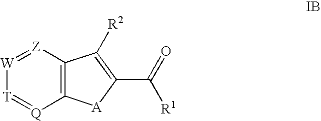
wherein
A represents oxygen, CH₂, or NR⁸;
and optical isomers, pharmaceutically acceptable salts, hydrates, solvates, and polymorphs thereof;

Such a pharmaceutical composition comprising, together with one or more pharmaceutically acceptable excipients or vehicles, a compound selected from those of formula IC 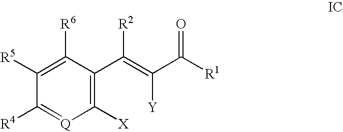
wherein
X represents hydrogen, C₁₋₆alkyl, halogen, cyano, C₁₋₆alkoxy, nitro, or di-(C₁₋₆alkyl)amino; and
Y represents hydrogen, halogen, cyano, C₁₋₆alkyl, C₁₋₆alkoxy, hydroxyC₁₋₆alkyl, or di-C₁₋₆alkylaminoC₁₋₆alkyl;
and optical isomers, pharmaceutically acceptable salts, hydrates, solvates, and polymorphs thereof.

Further, a kit for preliminary screening of candidate metabotropic glutamate receptor modulators for safety and efficacy, said kit comprising a compound of the invention and at least one investigational compound wherein the compound of the invention is used as a standard.

Specific compounds of formula I within the present invention include but are not limited to:
1-Cyclopropyl-3-(3-methoxyphenyl)-propenone
1-Adamantan-1-yl-3-(3-methoxyphenyl)-propenone
1-Cyclopropyl-3-(3,5-dimethoxy-phenyl)-propenone
1-Adamantan-1-yl-3-(3,5-dimethoxy-phenyl)-propenone
1-Cyclopropyl-3-quinolin-3-yl-propenone
4,4-Dimethyl-1-quinolin-3-yl-pent-1-en-3-one
1-(3,5-Dimethoxy-phenyl)4,4-dimethyl-pent-1-en-3-one
1-Adamantan-1-yl-3-(2,5-dimethoxy-phenyl)-propenone
1-Adamantan-1-yl-3-(4-methoxy-3-methyl-phenyl)-propenone
1-Adamantan-1-yl-3-(2,3-dihydrobenzo[1,4]dioxin-6-yl)-propenone
2-(Adamantane-1-carbonyl)-3-(2,3-dihydrobenzo[1,4]dioxin-6-yl)-acrylonitrile
1-Adamantan-1-yl-3-(3-benzyloxy-phenyl)-propenone
1-Adamantan-1-yl-3-(3,4,5-trimethoxy-phenyl)-propenone
1-(3-Methoxy-phenyl)-4,4-dimethyl-pent-1-en-3-one
Adamantan-1-yl-(2H-chromen-3-yl)-methanone
(6-Bromo-2H-chromen-3-yl)-phenylmethanone
Adamantan-1-yl-(7-methoxy-2H-chromen-3-yl)-methanone
Adamantan-1-yl-benzofuran-2-yl-methanone
Adamantan-1-yl-(7-ethoxy-benzofuran-2-yl)-methanone
Adamantan-1-yl-(5-methoxy-benzofuran-2-yl)-methanone
Benzofuran-2-yl-(2,5-dimethoxy-phenyl)-methanone
(2,5-Dimethoxy-phenyl)-(5-methoxy-benzofuran-2-yl)-methanone
(2,5-Dimethoxy-phenyl)-(6-methoxy-benzofuran-2-yl)-methanone
(2,5-Dimethoxy-phenyl)-(7-ethoxy-benzofuran-2-yl)-methanone
Adamantan-1-yl-(6-diethylamino-benzofuran-2-yl)-methanone
(6-Diethylamino-benzofuran-2-yl)-(3-methoxy-phenyl)-methanone
(6-Diethylamino-benzofuran-2-yl)-(2,5-dimethoxy-phenyl)-methanone
(6-Methoxy-benzofuran-2-yl)-(3-methoxy-phenyl)-methanone
(3,4-Dimethyl-phenyl)-(6-methoxy-benzofuran-2-yl)-methanone
Adamantan-1-yl-(5-bromo-benzofuran-2-yl)-methanone
Benzofuran-2-yl-(2,5-dimethoxy-phenyl)-methanone
Benzofuran-2-yl-(2,3-dihydrobenzo[1,4]dioxin-6-yl)-methanone
1-(6-Methoxy-benzofuran-2-yl)-2-methyl-2-phenyl-propan-1-one
Adamantan-1-yl-(5-nitro-benzofuran-2-yl)-methanone
Adamantan-1-yl-(4-methoxy-benzofuran-2-yl)-methanone
Adamantan-1-yl-(4-hydroxymethyl-7-methyl-furo[2,3-c]pyridin-2-yl)-methanone
Adamantan-1-yl-(6-methoxy-3-methyl-benzofuran-2-yl)-methanone
(6-Diethylamino-benzofuran-2-yl)-(2-nitro-phenyl)-methanone
Adamantan-1-yl-(6-fluoro-3-methyl-benzofuran-2-yl)-methanone
(6-Diethylamino-benzofuran-2-yl)-(2,3-dihydro-benzo[1,4]dioxin-6-yl)-methanone
(6-Diethylamino-benzofuran-2-yl)-p-tolyl methanone
4-(6-Diethylamino-benzofuran-2-carbonyl)-benzonitrile
(6-Diethylamino-benzofuran-2-yl)-(2,4-dimethyl-phenyl)-methanone
Adamantan-1-yl-(6-methoxy-benzofuran-2-yl)-methanone
2-[2-(4-Chloro-phenyl)-2-methyl-propionyl]-5-methoxy-indole-1-carboxylic acid diethylamide
2-(4-Chloro-phenyl)-1-(5-methoxy-1H-indol-2-yl)-2-methyl-propan-1-one
(5-Bromo-1-methyl-1H-indol-2-yl)-(4-fluoro-phenyl)-methanone
N-[2-(4-Fluoro-benzoyl)-1-methyl-1H-indol-5-yl]-acetamide
Adamantan-1-yl-(5-hydroxy-1H-indol-2-yl)-methanone
Adamantan-1-yl-(5-benzyloxy-1H-indol-2-yl)-methanone
(5-Benzyloxy-1H-indol-2-yl)-[1-(4-chloro-phenyl)-cyclopentyl]-methanone
2-(Adamantane-1-carbonyl)-1H-indole-5-carbonitrile
Adamantan-1-yl-(5-methoxy-1H-indol-2-yl)-methanone
[1-(4-Chloro-phenyl)-cyclopentyl]-(5-methoxy-1H-indol-2-yl)-methanone
(5-Bromo-1-methyl-1H-indol-2-yl)-p-tolyl-methanone
(5-Benzyloxy-1-methyl-1H-indol-2-yl)-(4-fluoro-phenyl)-methanone
(5-Benzyloxy-1-methyl-1H-indol-2-yl)-p-tolyl-methanone
N-[1-Methyl-2-(4-methyl-benzoyl)-1H-indol-5-yl]-acetamide
(5-Methoxy-1-methyl-1H-indol-2-yl)-p-tolyl-methanone
1-(5-Benzyloxy-1-methyl-1H-indol-2-yl)-2,2-dimethyl-propan-1-one
[1-(4-Chloro-phenyl)-cyclopentyl]-(6-fluoro-1H-indol-2-yl)-methanone
2-(4-Chloro-phenyl)-1-(6-fluoro-1H-indol-2-yl)-2-methyl-propan-1-one
Adamantan-1-yl-(6-fluoro-1H-indol-2-yl)-methanone
N-[2-(4-Fluoro-benzoyl)-1-methyl-1H-indol-5-yl]-N-methyl-acetamide
N-Methyl-N-[1-methyl-2-(4-methyl-benzoyl)-1H-indol-5-yl]-acetamide
Adamantan-1-yl-(5-fluoro-1H-indol-2-yl)-methanone
1-(5-Hydroxy-1-methyl-1H-indol-2-yl)-2,2-dimethyl-propan-1-one
Adamantan-1-yl-(1H-inden-2-yl)-methanone
(1H-Inden-2-yl)-(4-trifluoromethoxy-phenyl)-methanone
Adamantan-1-yl-(6-bromo-benzofuran-2-yl)-methanone
Adamantan-1-yl-(6-morpholin-4-yl-benzofuran-2-yl)-methanone
Adamantan-1-yl-(6-piperidin-1-yl-benzofuran-2-yl)-methanone
Adamantan-1-yl-(6-pyrrolidin-1-yl-benzofuran-2-yl)-methanone
Adamantan-1-yl-(6-pyridin-3-yl-benzofuran-2-yl)-methanone
Adamantan-1-yl-(6-amino-benzofuran-2-yl)-methanone
N-[2-(Adamantane-1-carbonyl)-benzofuran-6-yl]-acetamide
Adamantan-1-yl-(2H-pyrano[3,2-c]pyridin-3-yl)-methanone
Adamantan-1-yl-furo[3,2-c]pyridin-2-yl-methanone
Adamantan-1-yl-(7-bromo-2H-chromen-3-yl)-methanone
N-[3-(Adamantane-1-carbonyl)-2H-chromen-7-yl]-acetamide
Adamantan-1-yl-(7-dimethylamino-2H-chromen-3-yl)-methanone
Adamantan-1-yl-(7-pyrrolidin-1-yl-2H-chromen-3-yl)-methanone
Adamantan-1-yl-(7-piperidin-2H-chromen-3-yl)-methanone
Adamantan-1-yl-(7-morpholin-4-yl-2H-chromen-3-yl)-methanone
Adamantan-1-yl-[7-(4-methyl-piperazin-1-yl-2H-chromen-3-yl]-methanone
Adamantan-1-yl-(7-oxazol-2-yl-2H-chromen-3-yl]-methanone
Adamantan-1-yl-(7-thiazol-2-yl-2H-chromen-3-yl]-methanone
Adamantan-1-yl-(4,7-dimethyl-furo[2,3-c]pyridin-2-yl)-methanone
Adamantan-1-yl-(4-methoxymethyl-7-methyl-furo[2,3-c]pyridin-2-yl)-methanone
and optical isomers, pharmaceutically acceptable salts, hydrates, solvates, and polymorphs thereof.

### DETAILED DESCRIPTION OF THE INVENTION

For the purpose of the present invention, the carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, (C₁₋₃)alkyl refers to alkyl of one to three carbon atoms, inclusive, (i.e., methyl, ethyl, propyl, and isopropyl), straight and branched forms thereof.

As used herein, the term “C₁₋₆alkyl” represents straight or branched chain alkyl groups which may be optionally substitued by one or more substituents selected from halogen, trifluoromethyl, C₁₋₆alkoxy, amino, hydroxy, C₁₋₆alkylamino, and di-(C₁₋₆alkyl)amino. Examples of such alkyl groups include methyl, ethyl, n-propyl, 2-propyl, n-butyl, tert-butyl, —CF₃, —C₂F₅, —CBr₃ and —CCl₃. The term “C₂₋₆alkenyl” represents straight or branched chain alkenyl groups. The term C₁₋₆alkoxy represents straight or branched chain —O—C₁₋₆alkyl groups which may be optionally substituted by one or more substituents selected from halogen, trifluoromethyl, amino, hydroxy, C₁₋₆alkylamino and di-(C₁₋₆alkyl)amino. Examples of such alkoxy groups include methoxy, ethoxy, n-propoxy, i-propoxy, —OCF₃ and —OC₂F₅. The term “cycloC₃₋₁₂alkyl” represents monocyclic or bicyclic, or tricyclic alkyl groups, including cyclopropyl, cyclobutyl, cyclopentyl, cyclohexyl, bicyclo[2.2.1]heptyl and adamantanyl, which may be optionally substituted by one or more substituents, which may be the same or different, selected independently from halogen, trifluoromethyl, C₁₋₆alkyl, C₁₋₆alkoxy, amino, hydroxy, C₁₋₆alkylamino, and di-(C₁₋₆alkyl)amino. The term “aryl” signifies phenyl or naphthyl, or phenyl substituted by one or more substituents, which may be the same or different, selected from halogen, trifluoromethyl, trifluoromethoxy, C₁₋₆alkyl, C₂₋₆alkenyl, C₁₋₆alkoxy, amino, hydroxy, nitro, cyano, C₁₋₆alkoxycarbonyl, C₁₋₆alkylamino, di-(C₁₋₆alkyl)amino and C₁₋₆alkylenedioxy. The term “heteroaryl” represents an aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substitued by one or more substituents, which may be the same or different, selected from halogen, trifluoromethyl, C₁₋₆alkoxy, amino, hydroxy, nitro, cyano, C₁₋₆alkoxycarbonyl, C₁₋₆alkylamino, and di-(C₁₋₆alkyl)amino. Representative heteroaryl groups include furanyl, thienyl, pyrrolyl, oxazolyl, thiazolyl, imidazolyl, oxadiazolyl, tetrazolyl, pyridinyl, pyrimidinyl, pyridazinyl, benzofuryl, benzothienyl, indolyl, benzimidazolyl, benzoxazolyl, benzothiazolyl, quinolinyl and isoquinolinyl. The term “halogen” represents fluorine, chlorine, bromine and iodine.

The compounds of the present invention are named according to the IUPAC or CAS nomenclature system. Abbreviations which are well known to one of ordinary skill in the art may be used (e.g. “Ph” for phenyl, “Me” for methyl, “Et” for ethyl, “h” for hour or hours, and “rt” for room temperature).

The term “analog” or “derivative” is used herein in the conventional pharmaceutical sense, to refer to a molecule that structurally resembles a reference molecule, but has been modified in a targeted and controlled manner to replace one or more specific substituents of the referent molecule with an alternate substituent, thereby generating a molecule which is structurally similar to the reference molecule. Synthesis and screening of analogs (e.g., using structural and/or biochemical analysis), to identify slightly modified versions of a known compound which may have improved or biased traits (such as higher potency and/or selectivity at a specific targeted receptor type, greater ability to penetrate blood-brain barriers, fewer side effects, etc.) is a drug design approach that is well known in pharmaceutical chemistry.

In addition, using methods known to those skilled in the art, analogs and derivatives of the compounds of the invention can be created which have improved therapeutic efficacy, i.e., higher potency and/or selectivity at a specific targeted receptor type, either greater or lower ability to penetrate mammalian blood-brain barriers (e.g., either higher or lower blood-brain barrier permeation rate), fewer side effects, etc.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

Compounds of the present invention may be in the form of pharmaceutically acceptable salts. “Pharmaceutically acceptable salts” refers to those salts which possess the biological effectiveness and properties of the parent compound and which are not biologically or otherwise undesirable. The nature of the salt is not critical, provided that it is non-toxic and does not substantially interfere with the desired pharmacological activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, tautomeric, or stereoisomeric form, or mixture thereof, of a compound of the invention, which possesses the useful properties described herein.

The following Schemes 1-9 describe the preparation of compounds of formula I of the present invention. All of the starting materials are prepared by procedures described in these schemes, by procedures well known to one of ordinary skill in organic chemistry, or can be obtained commercially. All of the final compounds of the present invention are prepared by procedures described in these charts or by procedures analogous thereto, which would be well known to one of ordinary skill in organic chemistry. All of the variables used in the schemes are as defined below or as in the claims.

Compounds of formula I were obtained, as shown in Scheme 1. A compound of formula I is prepared from the corresponding amide 1 or nitrile 2 via reaction with an organometallic compound R¹-M as shown in Scheme 1. [Shimizu, Tomio; Hayashi, Yoshiyuki; Yamada, Kazunari; Nishio, Toshiyuki; Teramura, Kazuhiro; Bull. Chem. Soc. Jpn.; 54; 1; 1981; 217-222.] Derivatives of formula I are novel compounds and constitute a further aspect of the invention.

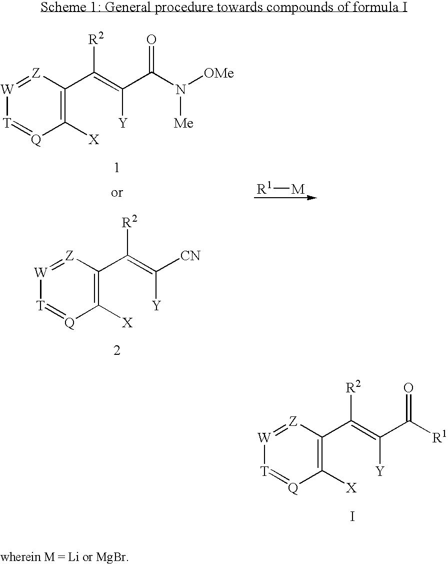

A compound of one embodiment of formula IA is prepared via Baylis-Hillman reaction (Scheme 2). [P. T. Kaye, X. Nocanda, J. Chem. Soc., Perkin Trans. 1, 2000, 1331-1332.] In this manner, 3A reacts with methyl vinyl ketone derivative 4 in the presence of the catalyst 1,4-diazabicyclo[2.2.2]octane (DABCO) via formation of intermediate 5 to give cyclic product IA. The same condensation may be accomplished via another mechanism if a strong base is used. In this case, a compound of the general formula IA is prepared by Michael addition of 3A with the methyl vinyl ketone derivative 4 followed by an intramolecular condensation of intermediate 6. The reaction may conveniently be effected by using an alkali metal hydroxide (e.g., sodium or potassium hydroxide) in an alcohol (e.g., methanol or ethanol) or water, or mixtures thereof, or using alkali metal alkoxide (e.g., sodium ethoxide ot potassium tert-butoxide) in the corresponding alcohol (e.g., ethanol or tert-butanol) or in an inert solvent such as an ether (e.g., tetrahydrofuran) at a temperature in the range of 0° C. to 100° C. [L. Rene, R. Royer, Europ. J. Med. Chem., 1975, 10, 72.]

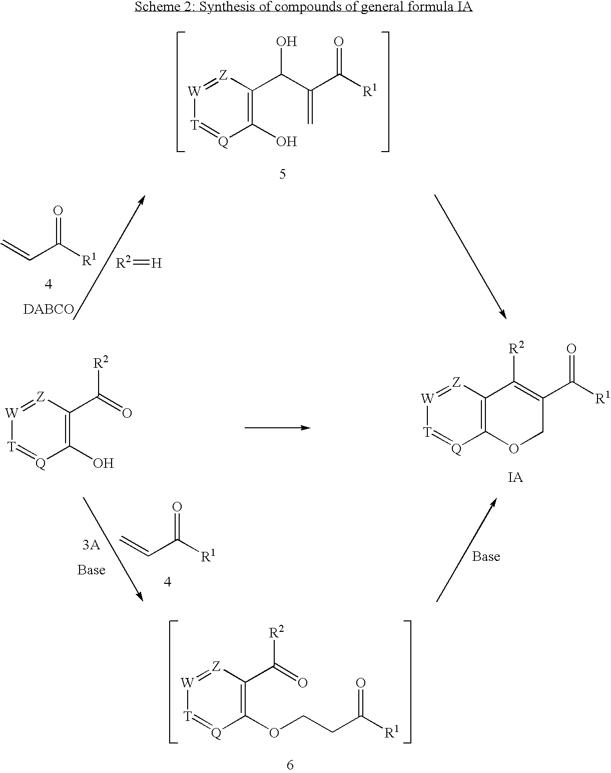

The condensation reaction between 3A and 4 or protected derivatives thereof may also be carried out in the presence of a base such as an alkali metal amide (e.g., lithium diisopropylamide) in an inert solvent such as an ether (e.g., tetrahydrofuran), followed by dehydration, and removal of any protecting group where necessary.

According to another general process (Scheme 3), a compound of general formula IA is prepared via the alkylation of a phenol derivative such as 3A by a beta-halo-ketone derivative 7 to give the intermediate 6 which may then be cyclized in the presence of a base.

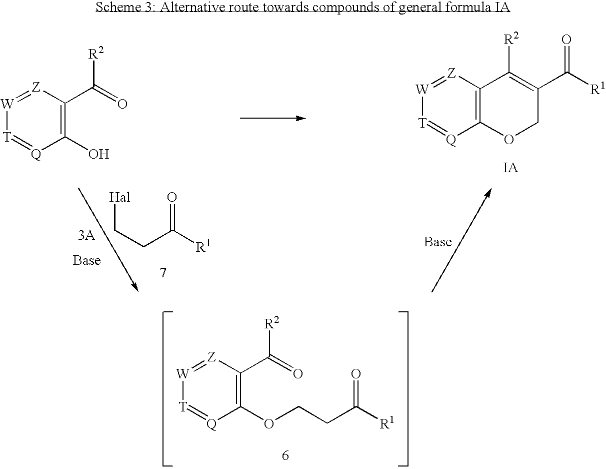

A compound of one embodiment of formula IB is prepared according to the procedure shown in Scheme 4. First, o-hydroxybenzaldehyde derivative 3A reacts with halo-ketone derivative 8A under basic conditions to give compound 9 which then undergoes a cyclization to form compound IB. The reaction may conveniently be effected by using an alkali metal hydroxide (e.g., sodium or potassium hydroxide) in an alcohol (e.g., methanol or ethanol) or water, or mixtures thereof, or using alkali metal alkoxide (e.g., sodium ethoxide or potassium tert-butoxide) in the corresponding alcohol (e.g., ethanol or tert-butanol) or in an inert solvent such as an ether (e.g., tetrahydrofuran) at a temperature in the range of 0 to 100° C. The condensation reaction between 3A and 8A or protected derivatives thereof may also be carried out in the presence of a base such as an alkali metal amide (e.g., lithium diisopropylamide) in an inert solvent such as ether (e.g., tetrahydrofuran), followed by dehydration, and removal of any protecting group where necessary. 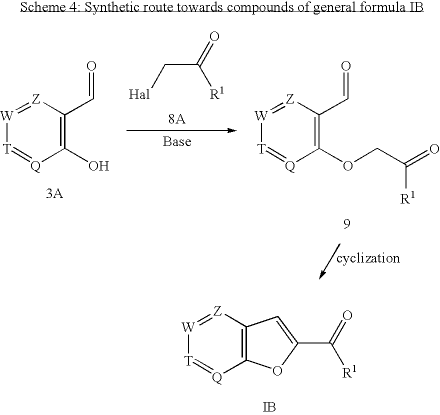
wherein Hal represents chlorine, bromine or iodine.

A compound of another embodiment of formula IB (A═NR⁸) is prepared according to the procedure shown in Scheme 5.

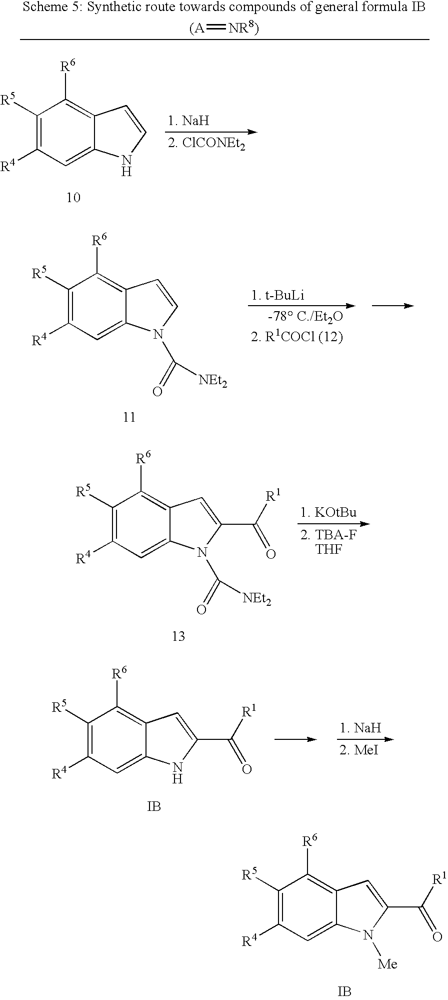

In addition to general reaction shown in Scheme 1, two additional approaches may be used to obtain ketones IB (A═NR⁸): 2-lithiation of corresponding indole derivatives 11 (Scheme 5) and addition of organometallics to indolyl-2-carboxylic acid chloride 17 (Scheme 6). The N,N-diethylcarbamoyl group was found to be an efficient lithiation directing group [Hartung, C. G.; Fecher, A.; Chapell, B.; Snieckus, V. Org. Lett. 2003, 5, 1899-1902.]. The combination of t-BuLi and Et₂O at −78° C. allows for a decrease in undesired side reactions and yields of ortho-lithiation are in the range of 35-75%. Cleavage of the lithiation directing group is readily accomplished with KOtBu in THF, followed with Bu₄NF. Another procedure is based on the reaction of arylmagnesium and arylzinc species with the appropriate 1H-indole-2-carbonyl chloride (Scheme 6).

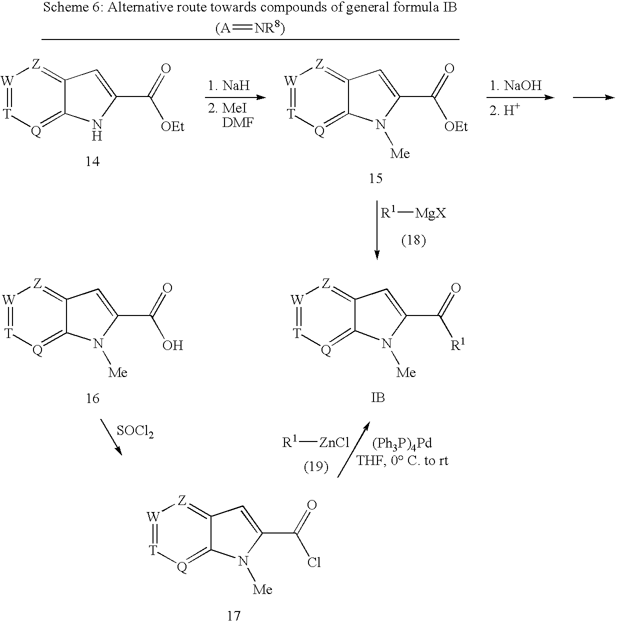

The addition of an arylmagnesium halide 18 to an ester 15 results in complex mixtures. This approach may be applied if the desired ketones IB are separable by flash chromatography. An ester 15 may also be converted to an acid chloride 17 and reacted with an arylzinc cloride 19 under Negishi conditions. Ketones IB may be thus obtained in 50-70% yield.

Amido- and amino-indolyl derivatives IB may also be synthesized from the corresponding parent bromoindole ketones 20 by a Cu-catalyzed C—N bond forming reaction [Klapars, A.; Huang, X.; Buchwald, S. L. J. Am. Chem. Soc. 2002, 124, 7421-7428.] as shown in Scheme 7.

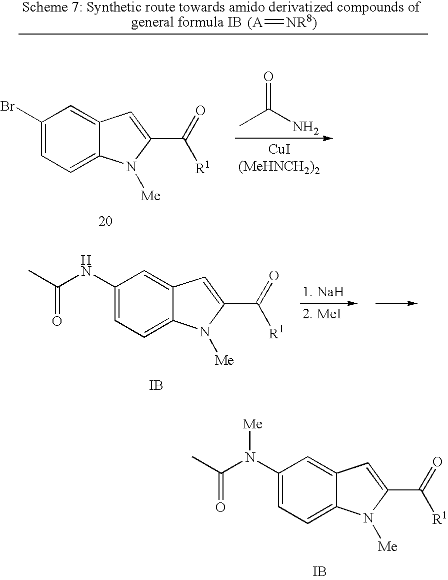

A compound of another embodiment of formula IB (A═CH₂) may be prepared according to the procedure shown in Error! Reference source not found. Generation of indenyl magnesium bromide 24 and subsequent reaction with an acid chloride 12 is an efficient [Ijpeij, E. G. Beijer, F. H.; Arts, H. J.; Newton, C.; de Vries, J. G.; Gruter, G. J. M. J. Org. Chem. 2002, 67, 169.] alternative to the Weinreb amide approach (Scheme 1) in preparing compounds IB (A═CH₂).

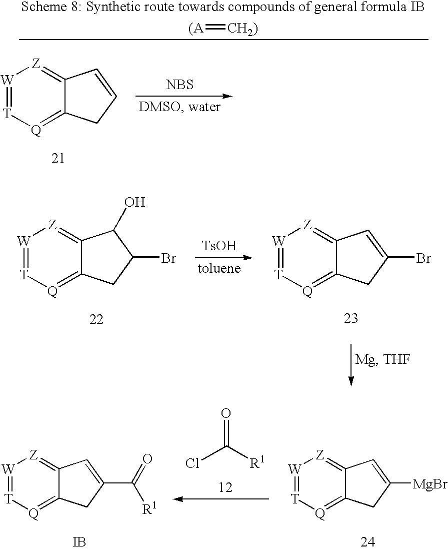

A compound of one embodiment of formula IC is prepared in condensation reaction of an aldehyde derivative such as 3 with an alkylketone under basic conditions according to the procedure shown in Scheme 9. The reaction may conveniently be effected by using an alkali metal hydroxide (e.g., sodium or potassium hydroxide) in an alcohol (e.g., methanol or ethanol) or water, or mixtures thereof, or using alkali metal alkoxide (e.g., sodium ethoxide ot potassium tert-butoxide) in the corresponding alcohol (e.g., ethanol or tert-butanol) or in an inert solvent such as an ether (e.g., tetrahydrofuran) at a temperature in the range of 0 to 100° C. The condensation reaction between 3 and 8 or protected derivatives thereof may also be carried out in the presence of a base such as an alkali metal amide (e.g., lithium diisopropylamide) in an inert solvent such as an ether (e.g., tetrahydrofuran), followed by dehydration, and removal of any protecting group where necessary.

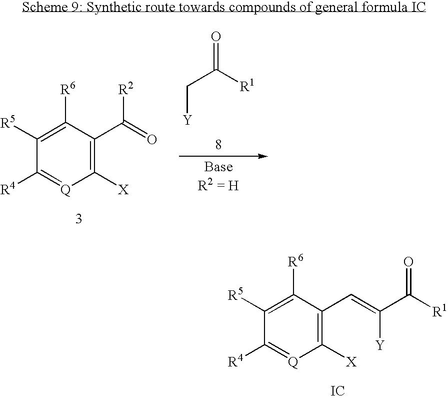

It will be appreciated that in the above transformations it may be necessary or desirable to protect any sensitive groups in the molecule of the compound in question in order to avoid undesirable side reactions.

It will be apparent to those skilled in the art that the described synthetic procedures are merely representative in nature and that alternative synthetic processes are known to one of ordinary skill in organic chemistry.

### EXPERIMENTAL PART

The compounds and their preparation of the present invention will be better understood in connection with the following examples, which are intended as an illustration of and not a limitation upon the scope of the invention.

Hereinafter, “DMF” is defined as N,N-dimethylformamide, “HCl” as hydrochloric acid, “DMSO” as dimethylsulfoxide and “TMS” as tetramethylsilane.

### Example 1

### 1-Cyclopropyl-3-(3-methoxyphenyl)-propenone

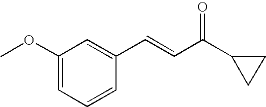

To a solution of 1-cyclopropylethanone (84.1 mg, 93.7 μl, 1 mmol) in ethanol (5 ml) is added 3-methoxybenzaldehyde (136.15 mg, 121.7 μl, 1 mmol) and 1N aqueous NaOH (1.3 ml). The mixture is stirred for 36 h, then brine (20 ml) is added and the mixture is extracted with dichloromethane (30 ml). The organic phase is washed with brine, dried over anhydrous potassium carbonate, filtered and concentrated under reduced pressure. The residue is purified by column chromatography (light petroleum ether:EtOAc, 10:1) to yield the title compound (143 mg, 70%) as a colorless oil.

Physical characteristics are as follows:

¹H NMR (CDCl₃, TMS) δ: 0.90-1.30, 2.26, 3.84, 6.86, 6.95, 7.09, 7.16, 7.32, 7.58.

### Example 2

### 1-Adamantan-1-yl-3-(3-methoxyphenyl)-propenone

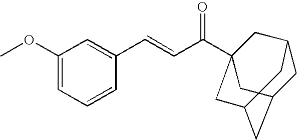

In close analogy to the procedure described in Example 1, 1-adamantan-1-yl-ethanone is reacted with 3-methoxybenzaldehyde to yield the title compound as a colorless oil that solidifies on standing.

Physical characteristics are as follows:

Mp 71-73° C.; ¹H NMR (CDCl₃, TMS) δ: 1.75, 1.88, 2.09, 3.84, 6.93, 7.08, 7.13, 7.17, 7.30, 7.63.

### Example 3

### 1-Cyclopropyl-3-(3,5-dimethoxy-phenyl)-propenone

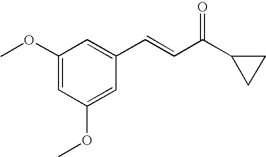

In close analogy to the procedure described in Example 1, 1-cyclopropylethanone is reacted with 3,5-dimethoxybenzaldehyde to yield the title compound as a pale yellow oil.

Physical characteristics are as follows:

¹H NMR (CDCl₃, TMS) δ: 0.97, 1.16, 2.26, 3.82, 6.50, 6.71, 6.83, 7.53.

### Example 4

### 1-Adamantan-1-yl-3-(3,5-dimethoxy-phenyl)-propenone

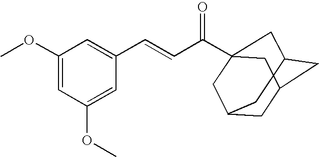

In close analogy to the procedure described in Example 1, 1-adamantan-1-yl-ethanone is reacted with 3,5-dimethoxybenzaldehyde to yield the title compound as a colorless solid.

Physical characteristics are as follows:

Mp 118-119° C.; ¹H NMR (CDCl₃, TMS) δ: 1.75, 1.88, 2.08, 3.82, 6.49, 6.70, 7.09, 7.58; Anal. Found (C₂₁H₂₆O₃) (%) C, 77.0; H, 8.0.

### Example 5

### 1-Cyclopropyl-3-quinolin-3-yl-propenone

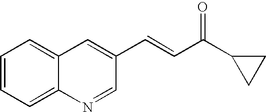

In close analogy to the procedure described in Example 1, 1-cyclopropylethanone is reacted with quinoline-3-carbaldehyde to give the title compound as a colorless solid.

Physical characteristics are as follows:

Mp 101-102° C.; ¹H NMR (CDCl₃, TMS) δ: 1.04, 1.22, 2.30, 7.11, 7.59, 7.77, 7.76, 7.86, 8.12, 8.29, 9.13; Anal. Found (C₁₅H₁₃NO) (%) C, 80.6; H, 5.8; N, 6.2.

### Example 6

### 4,4-Dimethyl-1-quinolin-3-yl-pent-1-en-3-one

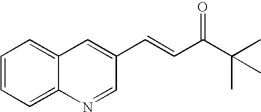

In close analogy to the procedure described in Example 1, 3,3-dimethylbutanone-2 is reacted with quinoline-3-carbaldehyde to yield the title compound as a colorless solid.

Physical characteristics are as follows:

Mp 146-148° C.; ¹H NMR (CDCl₃, TMS) δ: 1.27, 7.33, 7.59, 7.76, 7.83, 7.86, 8.11, 8.27, 9.13.

### Example 7

### 1-Adamantan-1-yl-3-quinolin-3-yl-propenone

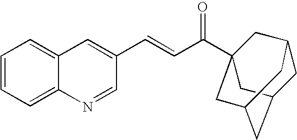

In close analogy to the procedure described in Example 1, 1-adamantan-1-yl-ethanone is reacted with quinoline-3-carbaldehyde to yield the title compound as a colorless solid.

Physical characteristics are as follows:

Mp 169-171° C.; ¹H NMR (CDCl₃, TMS) δ: 1.78, 1.93, 2.12, 7.37, 7.58, 7.75, 7.83, 7.87, 8.11, 8.27, 9.13; Anal. Found (C₂₂H₂₃NO) (%)C, 82.1; H, 7.3; N, 4.4.

### Example 8

### 1-(3,5-Dimethoxy-phenyl)-4,4-dimethyl-pent-1-en-3-one

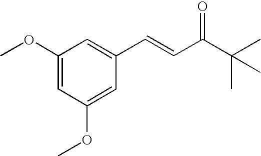

In close analogy to the procedure described in Example 1,3,3-dimethylbutan-2-one is reacted with 3,5-dimethoxybenzaldehyde to yield the title compound as a colorless oil.

Physical characteristics are as follows:

¹H NMR (CDCl₃, TMS) δ: 1.22, 3.82, 6.49, 6.70, 7.07, 7.59.

### Example 9

### 1-Adamantan-1-yl-3-(2,5 dimethoxy-phenyl)-propenone

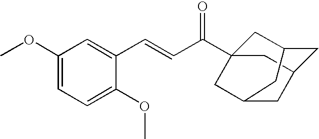

In close analogy to the procedure described in Example 1, 1-adamantan-1-yl-ethanone is reacted with 2,5-dimethoxybenzaldehyde to yield the title compound as a pale yellow solid.

Physical characteristics are as follows:

Mp 101-102° C.; ¹H NMR (CDCl₃, TMS) δ: 1.75, 1.88, 2.08, 3.81, 3.84, 6.84, 6.91, 7.10, 7.19, 7.95.

### Example 10

### 1-Adamantan-1-yl-3-(4-methoxy-3-methyl-phenyl)-propenone

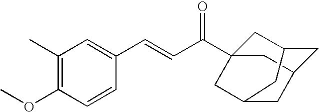

In close analogy to the procedure described in Example 1, 1-adamantan-1-yl-ethanone is reacted with 4-methoxy-3-methyl-benzaldehyde to yield the title compound as a colorless solid.

Physical characteristics are as follows:

Mp 138-140° C.; ¹H NMR (CDCl₃, TMS) δ: 1.76, 1.89, 2.09, 2.24, 3.86, 6.81, 7.02, 7.38, 7.40, 7.59; Anal. Found (C₂₁H₂₆O₂*0.5H₂O) (%) C, 78.3; H, 8.5.

### Example 11

### 1-Adamantan-1-yl-3-(2,3-dihydrobenzo[1,4]dioxin-6-yl)-propenone

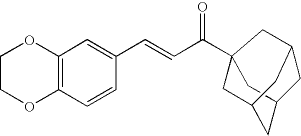

In close analogy to the procedure described in Example 1, 1-adamantan-1-yl-ethanone is reacted with 2,3-dihydro-benzo[1,4]dioxine-6-carbaldehyde to yield the title compound as a colorless solid.

Physical characteristics are as follows:

Mp 125-127° C.; ¹H NMR (CDCl₃, TMS) δ: 1.75, 1.87, 2.07, 4.28, 6.85, 7.00, 7.09, 7.11, 7.56; Anal. Found (C₂₁H₂₄O₃) (%) C, 77.3; H, 7.6.

### Example 12

### 1-Adamantan-1-yl-3-(3-benzyloxy-phenyl)-propenone

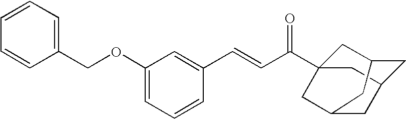

In analogy to the procedure described in Example 1, the title compound is obtained in moderate yield.

Physical characteristics are as follows:

Mp 100° C.; ¹H NMR (CDCl₃, TMS) δ: 1.76; 1.82, 2.09; 5.10; 7.00; 7.12; 7.15-7.20; 7.30-7.50; 7.62.

### Example 13

### 1-Adamantan-1-yl-3-(3,4,5-trimethoxy-phenyl)-propenone

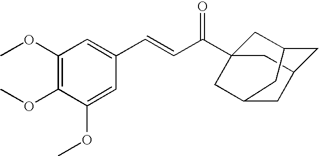

In analogy to the procedure described in Example 1, the title compound is obtained in moderate yield.

Physical characteristics are as follows:

Mp 160-161° C.; ¹H NMR (CDCl₃, TMS) δ: 1.77; 1.89, 2.10; 3.88; 3.92; 6.79; 7.02; 7.59.

### Example 14

### 1-(3-Methoxy-phenyl)-4,4-dimethyl-pent-1-en-3-one

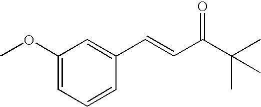

In analogy to the procedure described in Example 1, the title compound is obtained in moderate yield.

Physical characteristics are as follows:

¹H NMR (CDCl₃, TMS) δ: 1.22; 3.84; 3.84; 6.93; 7.0-7.5; 7.64.

### Example 15

### Adamantan-1-yl-(2H-chromen-3-yl)-methanone

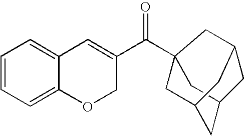

To a solution of 1-adamantan-1-yl-propenone (570 mg, 1 mmol) and 2-hydroxybenzaldehyde (366 mg, 1 mmol) in DMF (6 ml) is added NaH (60% oil dispersion, 120 mg, 3 mmol). The mixture is stirred under argon at 60° C. for 48 h. The reaction is quenched by addition of water and the mixture is extracted with ethyl acetate. The organic phase is washed with brine, dried over anhydrous magnesium sulfate, filtered and concentrated under reduced pressure. The residue is purified by column chromatography (hexane:EtOAc, 20:1) to yield the title compound as a colorless solid.

Physical characteristics are as follows:

¹H NMR (CDCl₃, TMS) δ: 1.72-1.78, 2.05-2.15, 4.90, 6.84, 6.93, 7.15, 7.23, 7.38.

### Example 16

### (6-Bromo-2H-chromen-3-yl)-phenylmethanone

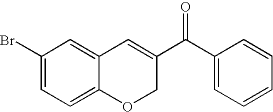

In analogy to the procedure described in Example 15, the title compound is obtained in moderate yield.

Physical characteristics are as follows:

Mp 128-130° C. (decomp.). ¹H NMR (CDCl₃, TMS) δ: 5.16; 6.79; 7.03; 7.22; 7.35; 7.45-7.60; 7.69-7.74.

### Example 17

### Adamantan-1-yl-(7-methoxy-2H-chromen-3-yl)-methanone

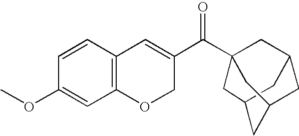

In analogy to the procedure described in Example 15, the title compound is obtained in moderate yield.

Physical characteristics are as follows:

Mp 174° C. (decomp.). ¹H NMR (CDCl₃, TMS) δ: 1.78; 2.05; 2.09; 3.80; 4.90; 6.41; 6.51; 7.07; 7.44.

### Example 18

### Adamantan-1-yl-benzofuran-2-yl-methanone

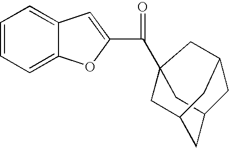

A solution of salicylaldehyde (122 mg, 1 mmol) and 1-adamantan-1-yl-2-bromo-ethanone (283 mg, 1.1 mmol) in 5 mL of ethanol is heated to reflux in the presence of powdered potassium hydroxide (79 mg, 1.4 mmol) for 14 h. The mixture is then evaporated to dryness and the residue is purified by column chromatography (dichloromethane) to yield 98 mg (35%) of the title compound as a colorless solid.

Physical characteristics are as follows:

Mp 129-131° C.; ¹H NMR (CDCl₃, TMS) δ: 1.82, 2.15, 7.27, 7.46, 7.54, 7.55, 7.69.

### Example 19

### Adamantan-1-yl-(7-ethoxy-benzofuran-2-yl)-methanone

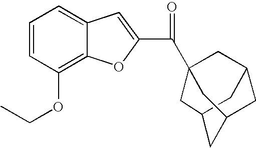

In analogy to the procedure described in Example 18, the title compound is obtained in moderate yield.

Physical characteristics are as follows:

Mp 84-86° C.; ¹H NMR (CDCl₃, TMS) δ:1.52; 1.81; 2.16; 4.32; 6.93; 7.17; 7.23; 7.50.

### Example 20

### Adamantan-1-yl-(5-methoxy-benzofuran-2-yl)-methanone

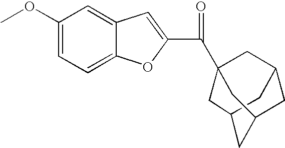

In analogy to the procedure described in Example 18, the title compound is obtained in moderate yield.

Physical characteristics are as follows:

Mp 113-115° C.; ¹H NMR (CDCl₃, TMS) δ: 1.81; 2.14; 3.85; 7.04-7.10; 7.43-7.48.

### Example 21

### Benzofuran-2-yl-(2,5-dimethoxy-phenyl)-methanone

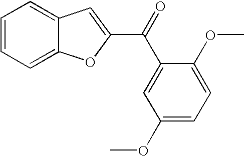

In analogy to the procedure described in Example 18, the title compound is obtained in moderate yield.

Physical characteristics are as follows:

¹H NMR (DMSO-D₆, TMS) δ: 3.68; 3.73; 7.04; 7.10; 7.16; 7.35; 7.52; 7.54; 7.72; 7.80.

### Example 22

### (2,5-Dimethoxy-phenyl)-(5-methoxy-benzofuran-2-yl)-methanone

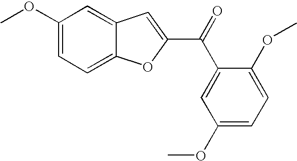

In analogy to the procedure described in Example 18, the title compound is obtained in moderate yield.

Physical characteristics are as follows:

Mp 119-121° C.; ¹H NMR (DMSO-d₆, TMS) δ: 3.67; 3.73; 3.77; 7.00-7.02; 7.10-7.16; 7.25; 7.43; 7.62.

### Example 23

### (2,5-Dimethoxy-phenyl)-(6-methoxy-benzofuran-2-yl)-methanone

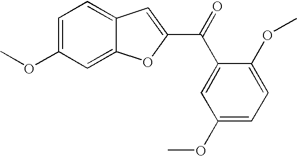

In analogy to the procedure described in Example 18, the title compound is obtained in moderate yield.

Physical characteristics are as follows:

Mp 129-131° C.; ¹H NMR (DMSO-d₆, TMS) δ: 3.68, 3.73; 3.83; 6.97; 6.99; 7.08; 7.13; 7.31; 7.42; 7.65.

### Example 24

### (2,5-Dimethoxy-phenyl)-(7-ethoxy-benzofuran-2-yl)-methanone

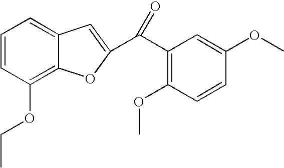

In analogy to the procedure described in Example 18, the title compound is obtained in moderate yield.

Physical characteristics are as follows:

Mp 55-57° C.; ¹H NMR (CDCl₃, TMS) δ: 1.50; 3.76; 3.81; 4.28; 6.92-6.98; 7.01-7.06; 7.14-7.22; 7.22; 7.32.

### Example 25

### Adamantan-1-yl-(6-diethylamino-benzofuran-2-yl)-methanone

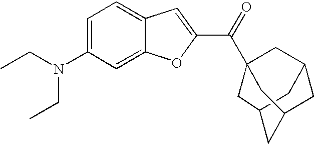

In analogy to the procedure described in Example 18, the title compound is obtained in moderate yield.

Physical characteristics are as follows:

Mp>160° C. (decomp.); ¹H NMR (CDCl₃, TMS) δ: 1.30; 1.83; 2.13; 3.38; 3.70; 7.52; 7.55; 7.86; 8.28.

### Example 26

### (6-Diethylamino-benzofuran-2-yl)-(3-methoxy-phenyl)-methanone

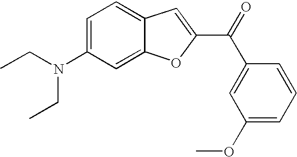

In analogy to the procedure described in Example 18, the title compound is obtained in moderate yield.

Physical characteristics are as follows:

Mp>135° C. (decomp.); ¹H NMR (CDCl₃, TMS) δ: 1.31; 3.54; 3.91; 7.19-7.24; 7.48; 7.57; 7.61; 7.71; 7.77; 7.90; 8.08.

### Example 27

### (6-Diethylamino-benzofuran-2-yl)-(2,5-dimethoxy-phenyl)-methanone

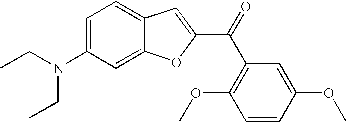

In analogy to the procedure described in Example 18, the title compound is obtained in moderate yield.

Physical characteristics are as follows:

Mp>125° C. (decomp.); ¹H NMR (CDCl₃, TMS) δ: 1.29; 3.49; 3.76; 3.82; 6.98; 7.03; 7.09; 7.43; 7.80; 7.86; 7.96.

### Example 28

### (6-Methoxy-benzofuran-2-yl)-(3-methoxy-phenyl)-methanone

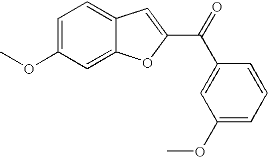

In analogy to the procedure described in Example 18, the title compound is obtained in moderate yield.

Physical characteristics are as follows:

Mp 63-65° C.; ¹H NMR (CDCl₃, TMS) δ: 3.89; 6.96; 7.11; 7.16; 7.43; 7.47; 7.50; 7.57; 7.60.

### Example 29

### (3,4-Dimethyl-phenyl)-(6-methoxy-benzofuran-2-yl)-methanone

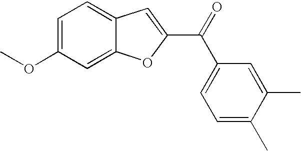

In analogy to the procedure described in Example 18, the title compound is obtained in moderate yield.

Physical characteristics are as follows:

Mp 95-97° C.; ¹H NMR (CDCl₃, TMS) δ: 2.36; 3.89; 6.96; 7.11; 7.27; 7.44; 7.57; 7.76; 7.78.

### Example 30

### Adamantan-1-yl-(5-bromo-benzofuran-2-yl)-methanone

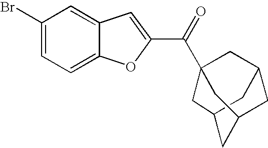

In analogy to the procedure described in Example 18, the title compound is obtained in moderate yield.

Physical characteristics are as follows:

Mp 115-117° C.; ¹H NMR (CDCl₃, TMS) δ: 1.81; 2.13; 7.45; 7.45; 7.54; 7.82.

### Example 31

### Benzofuran-2-yl-(2,3-dihydrobenzo[1,4]dioxin-6-yl)-methanone

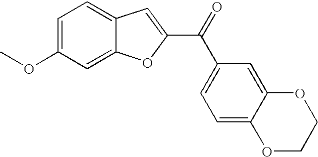

In analogy to the procedure described in Example 18, the title compound is obtained in moderate yield.

Physical characteristics are as follows:

Mp 68-70° C.; ¹H NMR (CDCl₃, TMS) δ: 3.89; 4.31-4.37; 6.95; 6.98; 7.10; 7.46; 7.57; 7.61; 7.64.

### Example 32

### 1-(6-Methoxy-benzofuran-2-yl)-2-methyl-2-phenyl-propan-1-one

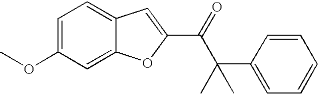

In analogy to the procedure described in Example 18, the title compound is obtained in moderate yield.

Physical characteristics are as follows:

Mp 93-95° C.; ¹H NMR (DMSO-d₆, TMS) δ: 2.50; 3.77; 6.87; 6.97; 7.11; 7.18-7.39; 7.51.

### Example 33

### Adamantan-1-yl-(5-nitro-benzofuran-2-yl)-methanone

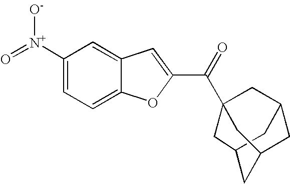

In analogy to the procedure described in Example 18, the title compound is obtained in moderate yield.

Physical characteristics are as follows:

Mp 151-153° C.; ¹H NMR (CDCl₃, TMS) δ: 1.83; 2.14; 7.64; 7.69; 8.38; 8.65.

### Example 34

### Adamantan-1-yl-(4-methoxy-benzofuran-2-yl)-methanone

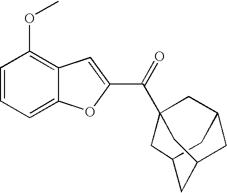

In analogy to the procedure described in Example 18, the title compound is obtained in moderate yield.

Physical characteristics are as follows:

Mp 96-98° C.; ¹H NMR (CDCl₃, TMS) δ: 1.81; 2.14; 3.96; 6.67; 7.17; 7.38; 7.64.

### Example 35

### Adamantan-1-yl-(4-hydroxymethyl-7-methyl-furo[2,3-c]pyridin-2-yl)-methanone hydrochloride

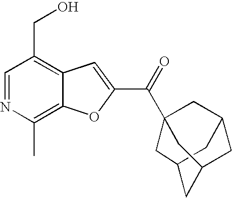

In analogy to the procedure described in Example 18, the title compound is obtained in moderate yield.

Physical characteristics are as follows:

Mp>120° C. (decomp.); ¹H NMR (CDCl₃, TMS) δ: 1.84; 2.12; 2.18; 3.19; 5.18; 7.75;

### Example 36

### Adamantan-1-yl-(6-methoxy-3-methyl-benzofuran-2-yl)-methanone

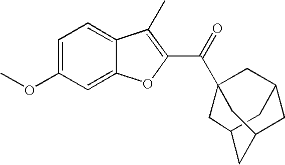

In analogy to the procedure described in Example 18, the title compound is obtained in moderate yield.

Physical characteristics are as follows:

Mp 173-175° C.; ¹H NMR (CDCl₃, TMS); δ; 1.81; 2.14; 2.54; 3.89; 6.92; 6.99; 7.49.

### Example 37

### (6-Diethylamino-benzofuro-2-yl)-(2-nitro-phenyl)-methanone

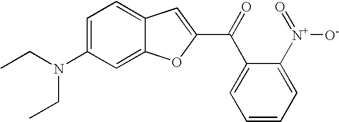

In analogy to the procedure described in Example 18, the title compound is obtained in moderate yield.

Physical characteristics are as follows:

Mp 173-175° C.; ¹H NMR (CDCl₃, TMS) δ: 1.81; 2.14; 2.54; 3.89; 6.92; 6.99; 7.49.

### Example 38

### Adamantan-1-yl-(6-fluoro-3-methyl-benzofuran-2-yl)-methanone

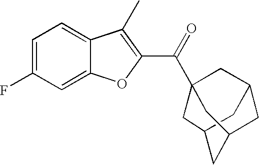

In analogy to the procedure described in Example 18, 1-(4-fluoro-2-hydroxy-phenyl)-ethanone is reacted with 1-adamantan-1-yl-2-bromo-ethanone to give the title compound in moderate yield.

Physical characteristics are as follows:

Mp 107-109° C.; ¹H NMR (CDCl₃, TMS) δ: 1.81; 2.13; 2.56; 7.07; 7.23; 7.57.

### Example 39

### (6-Diethylamino-benzofuran-2-yl)-(2,3-dihydro-benzo[1,4]dioxin-6-yl)-methanone

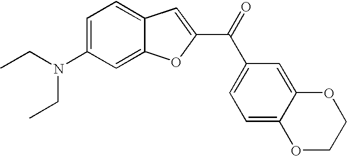

In analogy to the procedure described in Example 18, the title compound is obtained in moderate yield.

Physical characteristics are as follows:

Mp>110° C. (decomp.); ¹H NMR (CDCl₃, TMS) δ: 1.31; 3.55; 4.32-4.39; 7.02; 7.60; 7.71; 7.72-7.80; 7.90; 8.13.

### Example 40

### (6-Diethylamino-benzofuran-2-yl)-p-tolyl methanone

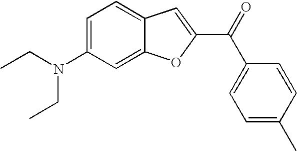

In analogy to the procedure described in Example 18, the title compound is obtained in moderate yield.

Physical characteristics are as follows:

Mp>115° C. (decomp.); ¹H NMR (CDCl₃, TMS) δ: 1.31; 2.48; 3.55; 7.36; 7.61; 7.75; 7.91; 8.04; 8.15.

### Example 41

### 4-(6-Diethylamino-benzofuran-2-carbonyl)-benzonitrile

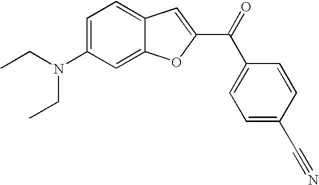

In analogy to the procedure described in Example 18, the title compound is obtained in moderate yield.

Physical characteristics are as follows:

Mp>100° C. (decomp.); ¹H NMR (CDCl₃, TMS) δ: 1.31; 3.56; 7.69; 7.71; 7.88; 7.99; 8.24; 8.26.

### Example 42

### (6-Diethylamino-benzofuran-2-yl)-(2,4-dimethyl-phenyl)-methanone

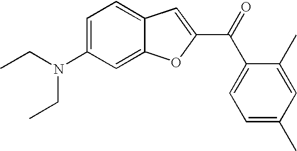

In analogy to the procedure described in Example 18, the title compound is obtained in moderate yield.

Physical characteristics are as follows:

Mp>110° C. (decomp.); ¹H NMR (CDCl₃, TMS) δ: 1.29; 2.41; 2.44; 3.53; 7.14; 7.16; 7.39; 7.54; 7.85; 7.98.

### Example 43

### Adamantan-1-yl-(6-methoxy-benzofuran-2-yl)-methanone

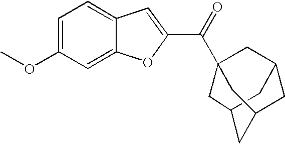

In analogy to the procedure described in Example 18, the title compound is obtained in moderate yield.

Physical characteristics are as follows:

Mp 99-101° C.; ¹H NMR (CDCl₃, TMS) δ: 1.81; 2.14; 3.88; 6.93; 7.05; 7.90; 7.53.

### Example 44

### 2-[2-(4-Chloro-phenyl)-2-methyl-propionyl]-5-methoxy-indole-1-carboxylic acid diethylamide

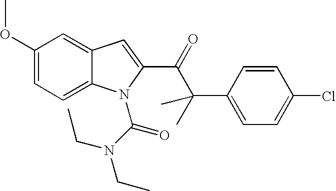

In a flame-dried and cooled under argon atmosphere reaction flask a solution of 5-methoxy-indole-1-carboxylic acid diethylamide (615 mg, 2.5 mmol) in diethyl ether (10 ml) is cooled under argon atmosphere to −78° C. and t-BuLi (1.77 ml of 1.5M soln., 2.65 mmol) is added within 20 min. The mixture is stirred for 1 h keeping the same temperature then a solution of 2-(4-chloro-phenyl)-2-methyl-propionyl chloride (1.356 g, 6.25 mmol) in diethyl ether is slowly added. The mixture is stirred for 2 h, then saturated aqueous ammonium chloride (5 ml) is added, the organic phase is separated and the aqueous phase is extracted with diethyl ether. The combined organic phases are washed with brine, dried over anhydrous sodium sulfate, filtered and concentrated under reduced pressure. The residue is purified by column chromatography (petroleum ether—EtOAc, 10:1) to yield the title compound (590 mg, 55%) as a colorless solid after recrystallization from petroleum ether—EtOAc.

Physical characteristics are as follows:

Mp 172-175° C.; ¹H NMR (DMSO-d₆, TMS) δ: 0.88-1.08, 1.18-1.40, 1.56, 2.91-3.18, 3.37-3.72, 3.69, 6.34, 6.98, 7.05, 7.16, 7.27-7.38, 7.39-7.49.

### Example 45

### 2-(4-Chloro-phenyl)-1-(5-methoxy-1H-indol-2-yl)-2-methyl-propan-1-one

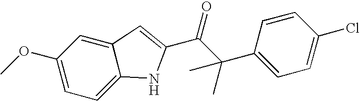

In a flame-dried reaction flask cooled under an argon atmosphere, 2-[2-(4-chloro-phenyl)-2-methyl-propionyl]-5-methoxy-indole-1-carboxylic acid diethylamide (426 mg, 1 mmol) is dissolved in dry THF (6 ml) and a TBAF (315.5 mg, 1 mmol) solution in THF (4 ml) is added, and the mixture is stirred for 1 h under argon atmosphere. Then a solution of KOtBu (247 mg, 2.2 mmol) in THF (5 ml) is added and the mixture is stirred for 2 h under an argon atmosphere. Saturated aqueous ammonium chloride (5 ml) is added and the resulting suspension is shaken with a water/EtOAc mixture, the organic phase is separated and the aqueous phase is extracted with EtOAc. The combined organic phases are washed with 1N aqueous HCl and brine, dried over anhydrous sodium sulfate, filtered and concentrated under reduced pressure. The residue is recrystallized from petroleum ether—EtOAc to yield the title compound (227 mg, 69%) as a colorless solid.

Physical characteristics are as follows:

Mp 177-179° C.; ¹H NMR (DMSO-d₆, TMS) δ: 1.57, 3.65, 6.14, 6.84, 6.91, 7.27, 7.29-7.45, 11.5.

### Example 46

### (5-Bromo-1-methyl-1H-indol-2-yl)-(4-fluoro-phenyl)-methanone

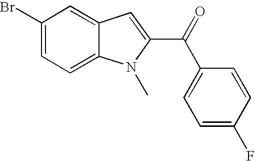

In a flame-dried reaction flask cooled under an argon atmosphere, 5-bromo-1-methyl-1H-indole-2-carbonyl chloride (200 mg, 0.73 mmol) (prepared according to the general procedure shown in Scheme 6) is added to tetrakis triphenylphosphine palladium (42.4 mg, 0.036 mmol), then dry THF (2 ml) is added. The mixture is cooled to 0° C. and a 4-fluorophenylzinc chloride solution (˜1.5 ml, ˜1.5 mmol; freshly prepared from ZnCl₂/TMEDA and 4-fluorophenylmagnesium bromide in THF) is slowly added via capillary and the mixture is stirred for 20 min. Then 1N aqueous HCl (3 ml) is added and the mixture is extracted with EtOAc, the organic phase is separated and the aqueous phase is extracted with EtOAc. The combined organic phases are washed with brine, dried over anhydrous sodium sulfate, filtered and concentrated under reduced pressure. The residue is purified by column chromatography (petroleum ether—EtOAc, 10:1) to yield the title compound (95 mg, 39%) as a colorless solid after recrystallization from petroleum ether —EtOAc.

Physical characteristics are as follows:

Mp 162-164° C.; ¹H NMR (DMSO-d₆, TMS) δ: 4.00, 6.98, 7.34-7.45, 7.49, 7.63, 7.90-8.00.

### Example 47

### N-[2-(4-Fluoro-benzoyl)-1-methyl-1H-indol-5-yl]-acetamide

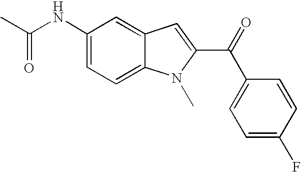

In a flame-dried reaction vial cooled an under argon atmosphere, (5-bromo-1-methyl-1H-indol-2-yl)-(4-fluoro-phenyl)-methanone (150 mg, 0.45 mmol) is mixed with acetamide (79.7 mg, 1.35 mmol), Cul (28.6 mg, 0.15 mmol) and K₃PO₄ (240 mg, 1.3 mmol), and dioxane (2.5 ml). The mixture is kept under an argon flow for 20 min then N,N′-dimethylethylenediamine (47.9 μl, 0.45 mmol) is added. The reaction vial is closed by teflon stopper and the mixture is heated overnight at 110° C. Then the reaction mixture is poured into water (50 ml) and extracted with EtOAc (3×40 ml). The combined organic phases are washed with aqueous NaHCO₃ and 0.1N aqueous HCl, water and brine, dried over anhydrous sodium sulfate, filtered and concentrated under reduced pressure to give the title compound (140 mg, 99%) as a colorless solid.

Physical characteristics are as follows:

Mp 213-215° C.; ¹H NMR (DMSO-d₆, TMS) δ: 2.05, 4.01, 7.00, 7.35-7.51, 7.58, 7.92-8.01, 8.05-8.07, 9.91.

### Example 48

### Adamantan-1-yl-(5-hydroxy-1H-indol-2-yl)-methanone

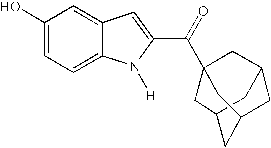

In analogy to the procedure described in Scheme 5, the title compound is obtained in moderate yield.

Physical characteristics are as follows:

Mp 197-199° C.; ¹H NMR (DMSO-d₆, TMS) δ: 1.69-1.82; 2.01-2.07; 6.77; 6.92; 7.22; 7.27; 8.6-9.6; 11.2.

### Example 49

### Adamantan-1-yl-(5-benzyloxy-1H-indol-2-yl)-methanone

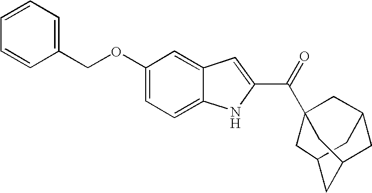

In analogy to the procedure described in Scheme 5, the title compound is obtained in moderate yield.

Physical characteristics are as follows:

Mp 189-191° C.; ¹H NMR (DMSO-d₆, TMS) δ: 1.73-1.77; 1.99-2.06; 5.07; 6.96; 7.14-7.16; 7.26-7.46; 11.3.

### Example 50

### (5-Benzyloxy-1H-indol-2-yl)-[1-(4-chloro-phenyl)-cyclopentyl]-methanone

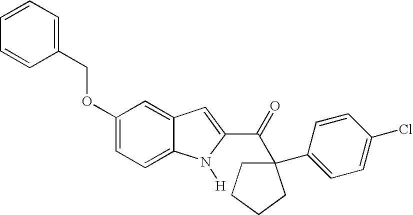

In analogy to the procedure described in Scheme 5, the title compound is obtained in moderate yield.

Physical characteristics are as follows:

Mp 134-136° C.; ¹H NMR (DMSO-d₆, TMS) δ: 1.55-1.77; 2.00-2.18; 2.38-2.58; 5.01; 6.44; 6.92; 7.01; 7.22-7.40; 11.5.

### Example 51

### (5-Benzyloxy-1H-indol-2-yl)-p-tolyl-methanone

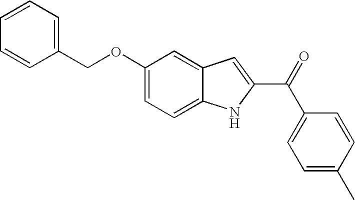

In analogy to the procedure described in Scheme 5, the title compound is obtained in moderate yield.

Physical characteristics are as follows:

Mp 199-200° C.; ¹H NMR (DMSO-d₆, TMS) δ: 2.41; 5.08; 6.99-7.00; 7.03; 7.20-7.23; 7.30-7.47; 7.78-7.84; 11.8.

### Example 52

### 2-(Adamantane-1-carbonyl)-1H-indole-5-carbonitrile

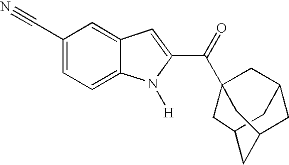

In analogy to the procedure described in Scheme 5, the title compound is obtained in moderate yield.

Physical characteristics are as follows:

Mp 250-252° C.; ¹H NMR (DMSO-d₆, TMS) δ: 1.75-1.80; 2.04-2.08; 7.55-7.57; 62-7.64; 8.22-8.24; 12.0.

### Example 53

### Adamantan-1-yl-(5-methoxy-1H-indol-2-yl)-methanone

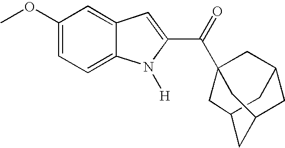

In analogy to the procedure described in Scheme 5, the title compound is obtained in moderate yield.

Physical characteristics are as follows:

Mp 229-231° C.; ¹H NMR (DMSO-d₆, TMS) δ: 1.68-1.84; 2.00-2.11; 3.74; 6.89; 7.10; 7.31; 7.37; 11.3.

### Example 54

### [1-(4-Chloro-phenyl)-cyclopentyl]-(5-methoxy-1H-indol-2-yl)-methanone

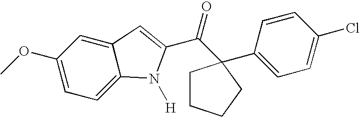

In analogy to the procedure described in Scheme 5, the title compound is obtained in moderate yield.

Physical characteristics are as follows:

Mp 157-159° C.; ¹H NMR (DMSO-d₆, TMS) δ: 1.53-1.77; 2.00-2.20; 2.39-2.60; 3.66; 6.45; 6.84; 6.93; 7.26; 7.30-7.42; 11.5.

### Example 55

### (4-Fluoro-phenyl)-(5-methoxy-1H-indol-2-yl)-methanone

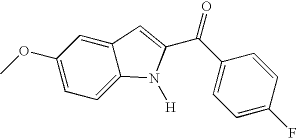

In analogy to the procedure described in Scheme 5, the title compound is obtained in moderate yield.

Physical characteristics are as follows:

Mp 167-169° C.; ¹H NMR (DMSO-d₆, TMS) δ: 3.75; 6.97; 7.00-7.04; 7.13; 7.34-7.46; 7.93-8.05; 11.8.

### Example 56

### (5-Bromo-1-methyl-1H-indol-2-yl)-p-tolyl-methanone

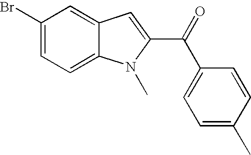

In analogy to the procedure described in Scheme 6, the title compound is obtained in moderate yield.

Physical characteristics are as follows:

Mp 169-171° C.; ¹H NMR (DMSO-d₆, TMS) δ: 2.41; 3.99; 6.95; 7.35-7.39; 7.47; 7.61; 7.74-7.80; 7.91.

### Example 57

### (5-Benzyloxy-1-methyl-1H-indol-2-yl)-(4-fluoro-phenyl)-methanone

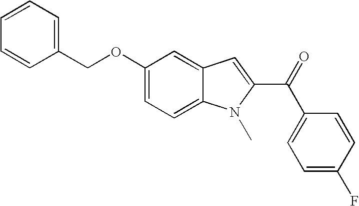

In analogy to the procedure described in Scheme 6, the title compound is obtained in moderate yield.

Physical characteristics are as follows:

Mp 118-120° C.; ¹H NMR (DMSO-d₆, TMS) δ: 4.00; 5.10; 6.89; 7.13; 7.23; 7.29-7.48; 7.55; 7.88-7.98.

### Example 58

### (5-Benzyloxy-1-methyl-H-indol-2-yl)-p-tolyl-methanone

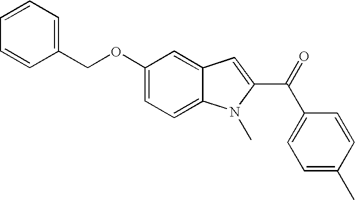

In analogy to the procedure described in Scheme 6, the title compound is obtained in moderate yield.

Physical characteristics are as follows:

Mp 132-134° C.: ¹H NMR (DMSO-d₆, TMS) δ: 2.40; 3.98; 5.09; 6.87; 7.12; 7.24; 7.30-7.48; 7.54; 7.73-7.79.

### Example 59

### N-[1-Methyl-2-(4-methyl-benzoyl)-1H-indol-5-yl]-acetamide

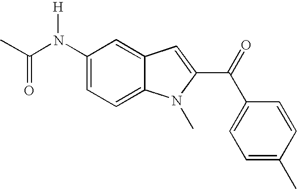

In analogy to the procedure described in Schemes 6 and 7, the title compound is obtained in moderate yield.

Physical characteristics are as follows:

Mp 211-213° C.: ¹H NMR (DMSO-d₆, TMS) δ: 2.05; 2.43; 4.00; 6.96; 7.34-7.41; 7.46; 7.56; 7.77-7.82; 8.05; 9.92.

### Example 60

### (5-Methoxy-1-methyl-1H-indol-2-yl)-p-tolyl-methanone

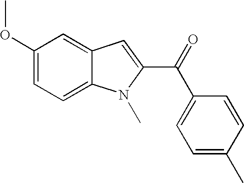

In analogy to the procedure described in Scheme 6, the title compound is obtained in moderate yield.

Physical characteristics are as follows:

Mp 149-151° C.: ¹H NMR (DMSO-d₆, TMS) δ: 2.40; 3.75; 3.98; 6.88; 7.03; 7.14; 7.33-7.37; 7.52; 7.73-7.79.

### Example 61

### 1-(5-Benzyloxy-1-methyl-1H-indol-2-yl)-2,2-dimethyl-propan-1-one

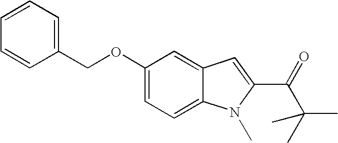

In analogy to the procedure described in Scheme 5, the title compound is obtained in moderate yield.

Physical characteristics are as follows:

Mp 101-103° C.; ¹H NMR (DMSO-d₆, TMS) δ: 1.32; 3.83; 5.10; 7.08; 7.23; 7.28-7.48.

### Example 62

### [1-(4-Chloro-phenyl)-cyclopentyl]-(6-fluoro-H-indol-2-yl)-methanone

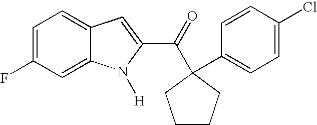

In analogy to the procedure described in Scheme 5, the title compound is obtained in moderate yield.

Physical characteristics are as follows:

Mp 143-145° C.; ¹H NMR (DMSO-d₆, TMS) δ: 1.59-1.74; 2.05-2.18; 2.44-2.56; 6.60-6.63; 6.81-6.92; 7.08; 7.32-7.42; 7.55; 11.7.

### Example 63

### 2-(4-Chloro-phenyl)-1-(6-fluoro-H-indol-2-yl)-2-methyl-propan-1-one

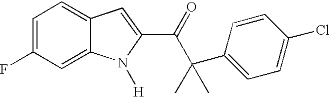

In analogy to the procedure described in Scheme 5, the title compound is obtained in moderate yield.

Physical characteristics are as follows:

Mp 172-174° C.; ¹H NMR (DMSO-d₆, TMS) δ: 1.58; 6.28-6.29; 6.78-6.89; 7.04-7.11; 7.30-7.36; 7.37-7.43; 7.51; 11.7.

### Example 64

### Adamantan-1-yl-(6-fluoro-1H-indol-2-yl)-methanone

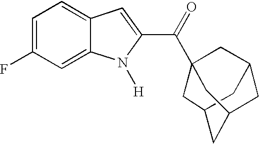

In analogy to the procedure described in Scheme 5, the title compound is obtained in moderate yield.

Physical characteristics are as follows:

Mp 242-244° C.; ¹H NMR (DMSO-d₆, TMS) δ: 1.71-1.84; 2.04-2.10; 6.94; 7.13; 7.52-7.54; 7.71; 11.6.

### Example 65

### N-[2-(4-Fluoro-benzoyl)-1-methyl-H-indol-5-yl]-N-methyl-acetamide

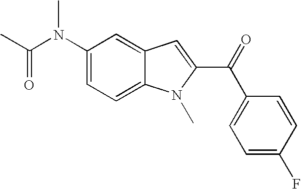

In analogy to the procedures described in Schemes 6 and 7, the title compound is obtained in moderate yield.

Physical characteristics are as follows:

Mp 155-157° C.; ¹H NMR (DMSO-d₆, TMS) δ: 1.73; 3.16; 4.04; 7.02; 7.31-7.46; 7.66; 7.71; 7.92-8.01.

### Example 66

### N-Methyl-N-[1-methyl-2-(4-methyl-benzoyl)-1H-indol-5-yl]-acetamide

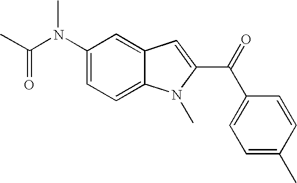

In analogy to the procedures described in Schemes 6 and 7, the title compound is obtained in moderate yield.

Physical characteristics are as follows:

Mp 193-195° C.; ¹H NMR (DMSO-d₆, TMS) δ: 1.73; 2.41; 3.16; 4.03; 6.99; 7.30-7.39; 7.65; 7.69; 7.76-7.82.

### Example 67

### Adamantan-1-yl-(5-fluoro-H-indol-2-yl)-methanone

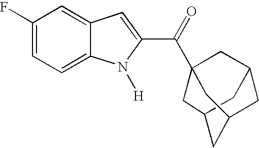

In analogy to the procedure described in Scheme 5, the title compound is obtained in moderate yield.

Physical characteristics are as follows:

Mp 223-225° C.; ¹H NMR (DMSO-d₆, TMS) δ: 1.69-1.83; 2.01-2.12; 7.10; 7.38-7.46; 11.6.

### Example 68

### 1-(5-Hydroxy-1-methyl-1H-indol-2-yl)-Z 2-dimethyl-propan-1-one

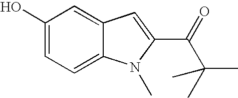

In analogy to the procedure described in Scheme 5, the title compound is obtained in moderate yield.

Physical characteristics are as follows:

Mp 126-128° C.; ¹H NMR (DMSO-d₆, TMS) δ: 1.33; 3.79; 6.82-6.89; 6.94-6.97; 7.21-7.24; 7.34; 8.99.

### Example 69

### Adamantan-1-yl-(1H-inden-2-yl)-methanone

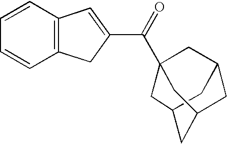 Adamantane-1-carbonyl chloride (993 mg, 5 mmol) is dissolved in dry THF (10 ml) and cooled under argon to −50° C. Then 1H-inden-2-ylmagnesium bromide (15 ml, ˜0.165M in THF, ˜2.5 mmol; prepared from 2-bromo-1H-indene; Scheme 8) is added and the cooling bath is removed after 15 min. The mixture is stirred for 1.5 h then it is treated by saturated aqueous ammonium chloride (50 ml) and extracted with diethyl ether (3×50 ml). The combined extracts are washed with saturated aqueous NaHCO₃ and brine, dried over anhydrous sodium sulfate, filtered and concentrated under reduced pressure. The residue is purified by column chromatography (petroleum ether—EtOAc, 50:1-30:1) to yield the title compound as a colorless solid after recrystallization from petroleum ether.
Physical characteristics are as follows:

Mp 124-125° C.; ¹H NMR (CDCl₃, TMS) δ: 1.76-1.84, 2.02-2.16, 3.69-3.75, 7.27-7.38, 7.42-7.59, 7.67-7.74.

### Example 70

### (1H-inden-2-yl)-(4-trifluoromethoxy-phenyl)-methanone

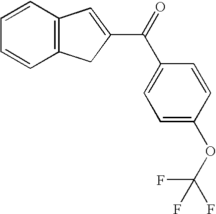

In analogy to the procedure described in Example 69, the title compound is obtained in moderate yield.

Physical characteristics are as follows:

Mp 115-117° C.; ¹H NMR (CDCl₃, TMS) δ: 3.85-3.90; 7.28-7.44; 7.47-7.61; 7.83-7.92.

### Example 71

### Adamantan-1-yl-(6-bromo-benzofuran-2-yl)-methanone

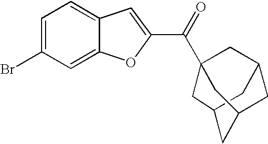

In analogy to the procedure described in Scheme 4, the title compound is obtained in moderate yield.

Physical characteristics are as follows:

Mp 128-130° C.; ¹H NMR (CDCl₃, TMS) δ: 1.81, 2.13, 7.42, 7.49, 7.55, 7.78.

### Example 72

### Adamantan-1-yl-(6-morpholin-4-yl-benzofuran-2-yl)-methanone

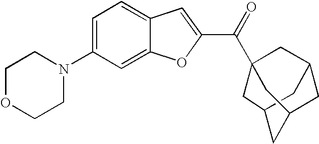

In analogy to the procedure described in Scheme 4, the title compound is obtained in moderate yield.

Physical characteristics are as follows:

Mp 123-125° C.; ¹H NMR (CDCl₃, TMS) δ: 1.81, 2.13, 3.24, 3.89, 6.97, 7.00, 7.46, 7.53.

### Example 73

### Adamantan-1-yl-(6-piperidin-1-yl-benzofuran-2-yl)-methanone

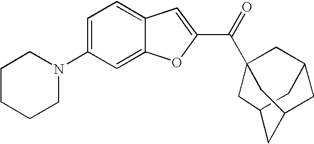

In analogy to the procedure described in Scheme 4, the title compound is obtained in moderate yield.

Physical characteristics are as follows:

Mp 150-152° C.; ¹H NMR (DMSO-d₆, TMS) δ: 1.57, 1.75, 2.02, 3.24-3.32, 7.05, 7.07, 7.54, 7.68.

### Example 74

### Adamantan-1-yl-(6-pyrrolidin-1-yl-benzofuran-2-yl)-methanone

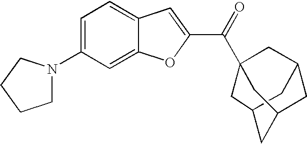

In analogy to the procedure described in Scheme 4, the title compound is obtained in moderate yield.

Physical characteristics are as follows:

Mp>45° C. (decomp.); ¹H NMR (CDCl₃, TMS) δ: 1.81, 2.02-2.09, 2.14, 3.36, 6.60, 6.63, 7.46, 7.46.

### Example 75

### Adamantan-1-yl-(6-pyridin-3-yl-benzofuran-2-yl)-methanone hydrochloride

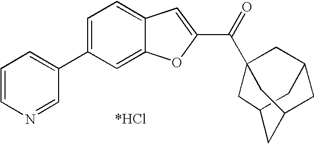

In analogy to the procedure described in Scheme 4, the title compound is obtained in moderate yield.

Physical characteristics are as follows:

Mp>120° C. (decomp.); ¹H NMR (DMSO-d₆, TMS) δ: 1.77, 2.07, 7.84, 7.93-7.99, 8.29, 8.47, 8.80, 9.25.

### Example 76

### Adamantan-1-yl-(6-amino-benzofuran-2-yl)-methanone hydrochloride

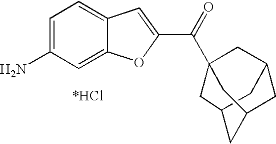

In analogy to the procedure described in Scheme 4, the title compound is obtained in moderate yield.

Physical characteristics are as follows:

Mp>150° C. (decomp.); ¹H NMR (DMSO-d₆, TMS) δ: 1.74, 2.01, 6.87, 7.00, 7.55, 7.74.

### Example 77

### N-[2-(Adamantane-1-carbonyl)-benzofuran-6-yl]-acetamide

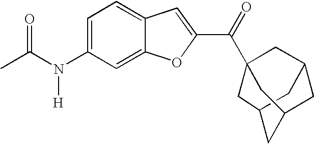

In analogy to the procedures described in Schemes 4 and 7, the title compound is obtained in moderate yield.

Physical characteristics are as follows:

Mp>145° C. (decomp.); ¹H NMR (CDCl₃, TMS) δ: 1.82, 2.14, 2.24, 7.10, 7.34, 7.48, 7.65, 8.20.

### Example 78

### Adamantan-1-yl-(2H-pyrano[3,2-c]pyridin-3-yl)-methanone

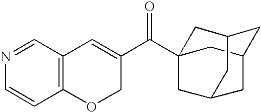

In analogy to the procedure described in Example 15, the title compound is obtained in moderate yield.

### Example 79

### Adamantan-1-yl-furo[3,2-c]pyridin-2-yl-methanone

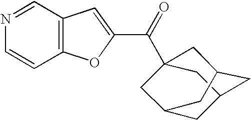

In analogy to the procedure described in Example 18, the title compound is obtained in moderate yield.

### Example 80

### Adamantan-1-yl-(7-bromo-2H-chromen-3-yl)-methanone

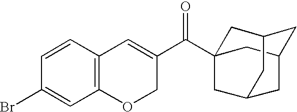

In analogy to the procedure described in Example 15, the title compound is obtained in moderate yield.

### Example 81

### N-[3-(Adamantane-1-carbonyl)-2H-chromen-7-yl]-acetamide

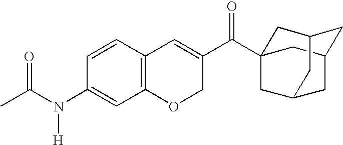

In analogy to the procedure described in Example 15, the title compound is obtained in moderate yield.

### Example 82

### Adamantan-1-yl-(7-dimethylamino-2H-chromen-3-yl)-methanone

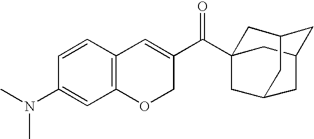

In analogy to the procedure described in Example 15, the title compound is obtained in moderate yield.

### Example 83

### Adamantan-1-yl-(7-pyrrolidin-1-yl-2H-chromen-3-yl)-methanone

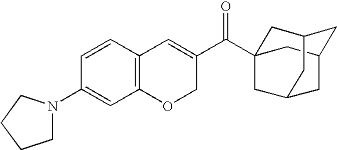

In analogy to the procedure described in Example 15, the title compound is obtained in moderate yield.

### Example 84

### Adamantan-1-yl-(7-piperidin-2H-chromen-3-yl)-methanone

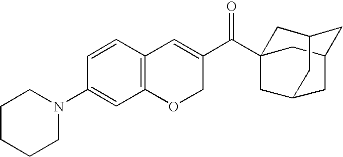

In analogy to the procedure described in Example 15, the title compound is obtained in moderate yield.

### Example 85

### Adamantan-1-yl-(7-morpholin-4-yl-2H-chromen-3-yl)-methanone

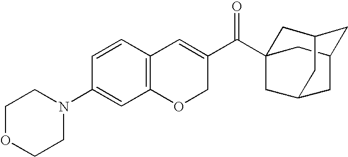

In analogy to the procedure described in Example 15, the title compound is obtained in moderate yield.

### Example 86

### Adamantan-1-yl-[7-(4-methyl-piperazin-1-yl-2H-chromen-3-yl]-methanone

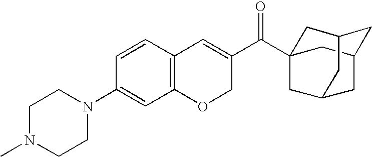

In analogy to the procedures described in Example 15 and Scheme 7, the title compound is obtained in moderate yield.

### Example 87

### Adamantan-1-yl-[7-oxazol-2-yl-2H-chromen-3-yl]-methanone

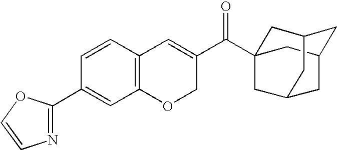

In analogy to the procedure described in Example 15, the title compound is obtained in moderate yield.

### Example 88

### Adamantan-1-yl-(7-thiazol-2-yl-2H-chromen-3-yl]-methanone

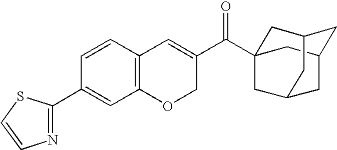

In analogy to the procedure described in Example 15, the title compound is obtained in moderate yield.

### Example 89

### Adamantan-1-yl-(4,7-dimethyl-furo[2,3-c]pyridin-2-yl)-methanone

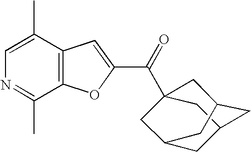

In analogy to the procedure described in Example 18, the title compound is obtained in moderate yield.

### Example 90

### Adamantan-1-yl-(4-methoxymethyl-7-methyl-furo[2,3-c]pyridin-2-yl)-methanone

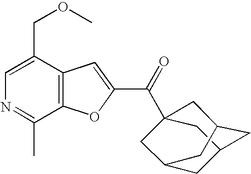

In analogy to the procedure described in Example 18, the title compound is obtained in moderate yield.

Pure stereoisomeric forms of the compounds and the intermediates of this invention may be obtained by the application of art-known procedures. Diastereomers may be separated by physical separation methods such as selective crystallization and chromatographic techniques, e.g. liquid chromatography using chiral stationary phases. Enantiomers may be separated from each other by selective crystallization of their diastereomeric salts with optically active acids. Alternatively, enantiomers may be separated by chromatographic techniques using chiral stationary phases. Said pure stereoisomeric forms may also be derived from the corresponding pure stereoisomeric form of appropriate starting materials, provided that the reaction occurs stereoselectively. Stereoisomeric forms of formula I are obviously intended to be included within the scope of this invention.

Addition Salts

For therapeutic use, salts of the compounds of formula I are those wherein the counterion is pharmaceutically acceptable. However, salts of acids and bases which are non-pharmaceutically acceptable may also find use, for example, in the preparation and purification of pharmaceutically acceptable compounds. All salts whether pharmaceutically acceptable or not are included within the ambit of the present invention. The pharmaceutically acceptable salts as mentioned above are meant to comprise the therapeutically active non-toxic salt forms which the compounds of formula I are able to form. The latter can conveniently be obtained by treating the base form with such appropriate acids as inorganic acids, e.g. hydrohalic acids such as hydrochloric, hydrobromic and the like; sulfuric acid; nitric acid; phosphoric acid and the like; or organic acids such as acetic, propanoic, hydroxyacetic, 2-hydroxypropanoic, oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfonic, 2-hydroxybenzoic, 4-amino-2-hydroxybenzoic and the like acids. Conversely, the salt form can be converted by treatment with alkali into the free base form.

Pharmaceutical Compositions

The active ingredients of the invention, together with one or more conventional adjuvants, carriers, or diluents, may be placed into the form of pharmaceutical compositions and unit dosages thereof, and in such form may be employed as solids, such as coated or uncoated tablets or filled capsules, or liquids, such as solutions, suspensions, emulsions, elixirs, or capsules filled with the same, all for oral use; in the form of suppositories or capsules for rectal administration or in the form of sterile injectable solutions for parenteral (including intravenous or subcutaneous) use. Such pharmaceutical compositions and unit dosage forms thereof may comprise conventional or new ingredients in conventional or special proportions, with or without additional active compounds or principles, and such unit dosage forms may contain any suitable effective amount of the active ingredient commensurate with the intended daily dosage range to be employed.

The term “carrier” applied to pharmaceutical compositions of the invention refers to a diluent, excipient, or vehicle with which an active compound is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by E. W. Martin, 18^{th} Edition.

Method Of Treating

Due to their high degree of activity and their low toxicity, together presenting a most favorable therapeutic index, the active principles of the invention may be administered to a subject, e.g., a living animal (including a human) body, in need thereof, for the treatment, alleviation, or amelioration, palliation, or elimination of an indication or condition which is susceptible thereto, or representatively of an indication or condition set forth elsewhere in this application, preferably concurrently, simultaneously, or together with one or more pharmaceutically-acceptable excipients, carriers, or diluents, especially and preferably in the form of a pharmaceutical composition thereof, whether by oral, rectal, or parental (including intravenous and subcutaneous) or in some cases even topical route, in an effective amount. Suitable dosage ranges may be identified through routine experimentation, depending as usual upon the exact mode of administration, form in which administered, the indication toward which the administration is directed, the subject involved and the body weight of the subject involved, and the preference and experience of the physician or veterinarian in charge.

The term “therapeutically effective” applied to dose or amount refers to that quantity of a compound or pharmaceutical composition that is sufficient to result in a desired activity upon administration to a living animal body in need thereof.

The active agents of the present invention may be administered orally, topically, parenterally, or mucosally (e.g., buccally, by inhalation, or rectally) in dosage unit formulations containing conventional non-toxic pharmaceutically acceptable carriers. It is usually desirable to use the oral route. The active agents may be administered orally in the form of a capsule, a tablet, or the like (see Remington's Pharmaceutical Sciences, Mack 5 Publishing Co., Easton, Pa.). The orally administered medicaments may be administered in the form of a time-controlled release vehicle, including diffusion-controlled systems, osmotic devices, dissolution-controlled matrices, and erodible/degradable matrices.

For oral administration in the form of a tablet or capsule, the active drug component can be combined with a non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like. For oral administration in liquid form, the drug components can be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) can also be added to stabilize the dosage forms.

The tablets can be coated by methods well known in the art. The compositions of the invention can be also introduced in microspheres or microcapsules, e.g., fabricated from polyglycolic acid/lactic acid. Liquid preparations for oral administration can take the form of, for example, solutions, syrups, emulsions or suspensions, or they can be presented as a dry product for reconstitution with water or other suitable vehicle before use. Preparations for oral administration can be suitably formulated to give controlled or postponed release of the active compound.

The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines, as is well known.

Drugs of the invention may also be delivered by the use of monoclonal antibodies as individual carriers to which the compound molecules are coupled. Active drugs may also be coupled with soluble polymers as targetable drug carriers. Such polymers can include polyvinyl-pyrrolidone, pyran copolymer, polyhydroxy-propyl methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

For administration by inhalation, the therapeutics according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The formulations of the invention can be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection can be presented in unit dosage form, e.g., in ampoules or in multi-dose containers, with an added preservative. The compositions can take such forms as excipients, suspensions, solutions, or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water, before use.

Compositions of the present invention can also be formulated for rectal administration, e.g., as suppositories or retention enemas (e.g., containing conventional suppository bases such as cocoa butter or other glycerides).

The compositions may, if desired, be presented in a pack or dispenser device which may contain one or more unit dosage forms containing the active ingredient, optionally at various dosage levels to act as a titration pack. The pack may, for example, comprise metal or plastic foil, such as a blister pack. The pack or dispenser device may be accompanied by instructions for administration. Compositions of the invention formulated in a compatible pharmaceutical carrier may also be prepared, placed in an appropriate container, and labeled for treatment of an indicated condition.

As disclosed herein, the dose of the components in the compositions of the present invention is determined to ensure that the dose administered continuously or intermittently will not exceed an amount determined after consideration of the results in test animals and the individual conditions of a patient. A specific dose naturally varies depending on the dosage procedure, the conditions of a patient or a subject animal such as age, body weight, sex, sensitivity, feed, dosage period, drugs used in combination, seriousness of the disease. The appropriate dose and dosage times under certain conditions can be determined by the test based on the above-described indices but may be refined and ultimately decided according to the judgment of the practitioner and each patient's circumstances (age, general condition, severity of symptoms, sex, etc.) according to standard clinical techniques.

Toxicity and therapeutic efficacy of the compositions of the invention can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it can be expressed as the ratio ED₅₀/LD₅₀. Compositions that exhibit large therapeutic indices are preferred.

### EXAMPLES OF REPRESENTATIVE PHARMACEUTICAL COMPOSITIONS

With the aid of commonly used solvents, auxiliary agents and carriers, the reaction products can be processed into tablets, coated tablets, capsules, drip solutions, suppositories, injection and infusion preparations, and the like and can be therapeutically applied by the oral, rectal, parenteral, and additional routes.

Representative pharmaceutical compositions follow.

(a) Tablets suitable for oral administration which contain the active ingredient may be prepared by conventional tabletting techniques.

(b) For suppositories, any usual suppository base may be employed for incorporation thereinto by usual procedure of the active ingredient, such as a polyethyleneglycol which is a solid at normal room temperature but which melts at or about body temperature.

(c) For parental (including intravenous and subcutaneous) sterile solutions, the active ingredient together with conventional ingredients in usual amounts are employed, such as for example sodium chloride and double-distilled water q.s., according to conventional procedure, such as filtration, aseptic filling into ampoules or IV-drip bottles, and autoclaving for sterility.

Other suitable pharmaceutical compositions will be immediately apparent to one skilled in the art.

### FORMULATION EXAMPLES

The following examples are given by way of illustration only and are not to be construed as limiting.

### Example 1

Tablet Formulation

A suitable formulation for a tablet containing 10 milligrams of active ingredient is as follows:

| | mg |
|---|---|
| Active Ingredient | 10 |
| Lactose | 61 |
| Microcrystalline Cellulose | 25 |
| Talcum | 2 |
| Magnesium stearate | 1 |
| Colloidal silicon dioxide | 1 |

### Example 2

### Tablet Formulation

Another suitable formulation for a tablet containing 100 mg is as follows:

| | mg |
|---|---|
| Active Ingredient | 100 |
| Polyvinylpyrrolidone, crosslinked | 10 |
| Potato starch | 20 |
| Polyvinylpyrrolidone | 19 |
| Magnesium stearate | 1 |
| Microcrystalline Cellulose | 50 |
| Film coated and colored. | |
| The film coating material consists of: | |
| Hypromellose | 10 |
| Microcryst. Cellulose | 5 |
| Talcum | 5 |
| Polyethylene glycol | 2 |
| Color pigments | 5 |

### Example 3

### Capsule Formulation

A suitable formulation for a capsule containing 50 milligrams of active ingredient is as follows:

| | mg |
|---|---|
| Active Ingredient | 50 |
| Corn starch | 26 |
| Dibasic calcium phosphate | 50 |
| Talcum | 2 |
| Colloidal silicon dioxide | 2 |
| filled in a gelatin capsule. | |

### Example 4

### Solution for Injection

A suitable formulation for an injectable solution is as follows:

| | | |
|---|---|---|
| Active Ingredient | mg | 10 |
| Sodium chloride | mg | q.s. |
| Water for Injection | mL | add 1.0 |

### Example 5

### Liquid Oral Formulation

A suitable formulation for 1 liter of an oral solution containing 2 milligrams of active ingredient in one milliliter of the mixture is as follows:

mg

| Active Ingredient | 2 | |
|---|---|---|
| Saccharose | 250 | |
| Glucose | 300 | |
| Sorbitol | 150 | |
| Orange flavor | 10 | |
| Colorant | q.s. | |
| Purified water | add 1000 | mL |

### Example 6

### Liquid Oral Formulation

Another suitable formulation for 1 liter of a liquid mixture containing 20 milligrams of active ingredient in one milliliter of the mixture is as follows:

| | G | |
|---|---|---|
| Active Ingredient | 20.00 | |
| Tragacanth | 7.00 | |
| Glycerol | 50.00 | |
| Saccharose | 400.00 | |
| Methylparaben | 0.50 | |
| Propylparaben | 0.05 | |
| Black currant-flavor | 10.00 | |
| Soluble Red color | 0.02 | |
| Purified water | add 1000 | mL |

### Example 7

### Liquid Oral Formulation

Another suitable formulation for 1 liter of a liquid mixture containing 2 milligrams of active ingredient in one milliliter of the mixture is as follows:

| | G | |
|---|---|---|
| Active Ingredient | 2 | |
| Saccharose | 400 | |
| Bitter orange peel tincture | 20 | |
| Sweet orange peel tincture | 15 | |
| Purified water | add 1000 | mL |

### EXAMPLE 8

### Aerosol Formulation

180 g aerosol solution contain:

| | G |
|---|---|
| Active Ingredient | 10 |
| Oleic acid | 5 |
| Ethanol | 81 |
| Purified Water | 9 |
| Tetrafluoroethane | 75 |

15 ml of the solution are filled into aluminum aerosol cans, capped with a dosing valve, purged with 3.0 bar.

### Example 9

### TDS Formulation

100 g solution contain:

| | G |
|---|---|
| Active Ingredient | 10.0 |
| Ethanol | 57.5 |
| Propyleneglycol | 7.5 |
| Dimethylsulfoxide | 5.0 |
| Hydroxyethylcellulose | 0.4 |
| Purified water | 19.6 |

1.8 ml of the solution are placed on a fleece covered by an adhesive backing foil. The system is closed by a protective liner which will be removed before use.

### Example 10

### Nanoparticle Formulation

10 g of polybutylcyanoacrylate nanoparticles contain:

| | G |
|---|---|
| Active Ingredient | 1.00 |
| Poloxamer | 0.10 |
| Butylcyanoacrylate | 8.75 |
| Mannitol | 0.10 |
| Sodium chloride | 0.05 |

Polybutylcyanoacrylate nanoparticles are prepared by emulsion polymerization in a water/0.1 N HCl/ethanol mixture as polymerizsation medium. The nanoparticles in the suspension are finally lyophilized under vacuum.

### PHARMACOLOGY—SUMMARY

The active principles of the present invention, and pharmaceutical compositions thereof and method of treating therewith, are characterized by unique advantageous and unpredictable properties, rendering the “subject matter as a whole”, as claimed herein, unobvious. The compounds and pharmaceutical compositions thereof exhibit, in standard accepted reliable test procedures, the following valuable properties and characteristics:

### Methods

### Binding Assays for the Characterization of MGLUR5 Modulator Properties

[³H]MPEP (2-methyl-6-(phenylethynyl)pyridine) binding to transmembrane allosteric modulatory sites of mGluR5 receptors in cortical/cerebellar membranes

Methods:

Preparation of Rat Cortical/Cerebellar Membranes:

Male Sprague-Dawley rats (200-250 g) are decapitated and their brains are removed rapidly. The cortex or cerebellum is dissected and homogenized in 20 volumes of ice-cold 0.32 M sucrose using a glass-Teflon homogenizer. The homogenate is centrifuged at 1000×g for 10 min. The pellet is discarded and the supernatant centrifuged at 20,000×g for 20 min. The resulting pellet is re-suspended in 20 volumes of distilled water and centrifuged for 20 min at 8000×g. Then the supernatant and the buffy coat are centrifuged at 48,000×g for 20 min in the presence of 50 mM Tris-HCl, pH 8.0. The pellet is then re-suspended and centrifuged two to three more times at 48,000×g for 20 min in the presence of 50 mM Tris-HCl, pH 8.0. All centrifugation steps are carried out at 4° C. After resuspension in 5 volumes of 50 mM Tris-HCl, pH 8.0 the membrane suspension is frozen rapidly at −80° C. 2276 of 13.6 nM is determined by Scatchard analysis and used according to the Cheng Prussoff relationship to calculate the affinity of displacers as Kd values (IC₅₀ of cold MRZ 2776 equates to a Ki of 13.7 nM). Bₘₐₓ was 0.56 pm/mg protein.

### Functional Assay of MGLUR1 Receptors in Cerebellar Granule Cells—Radioactive Assay for Changes in IP3 Levels

Preparation of Cerebellar Granule Cells

Cerebellar cortici are obtained from P8 postnatal Sprague Dawley rats, mechanically disrupted into small pieces with forceps and then transferred to Ca²⁺ and Mg²⁺ free Hank's buffered salt solution (HBSS-CMF) on ice. After three washes in HBSS-CMF, the tissue pieces are incubated 37° C. for 8 minutes in the presence of 0.25% trypsin/0.05% DNase. The enzymatic reaction is stopped with 0.016% DNAase/0.1% ovomucoid before centrifugation at 800 rpm for 5 minutes. The supernatant is replaced twice with NaHCO₃/HEPES-buffered basal Eagle medium (BME) plus 20 mM KCl. Cells are mechanically dissociated in 2 ml of BME by trituration through three Pasteur pipettes of successively decreasing tip diameter and then filtered through a 48 μM gauge filter. Cells are plated at a density of 150,000 cells in 50 μl in each well of poly-L-Lysin pre-coated 96 well plates (Falcon). The cells are nourished with BEM supplemented with 10% foetal calf serum, 2 mM glutamine (Biochrom), 20 mM KCl and gentamycin (Biochrom) and incubated at 36° C. with 5% CO₂ at 95% humidity. After 24 h, cytosine-β-D-arabinofuranoside (AraC, 10 μM) is added to the medium.

IP₃ Assay with [³H]myo-Inositol

After 6 DIV the culture medium is replaced completely with inositol free DMEM (ICN) containing [³H]myo-inositol (Perkin Elmer) at a final concentration of 0.5 μCi/100 μl/well and incubated for a further 48 hours. The culture medium in each well is replaced with 100 μL Locke's buffer (contains in (mM) NaCl (156), KCl (5.6), NaHCO₃ (3.6), MgCl₂ (1.0), CaCl₂ (1.3), Glucose (5.6), HEPES (10)) with additional (20 mM Li, pH 7.4) and incubated for 15 min at 37° C. Locke's buffer was replaced with agonists/agonists/putative mGluR1 ligands in Locke's buffer and incubated for 45 min. These solutions are then replaced by 100 μL 0.1M HCl in each well and incubated for a further 10 mins on ice. The 96 well plates can be frozen at −20° C. at this stage until further analysis.

Home made resin exchange columns are prepared as follows. Empty Bio-Spin Chromatography columns (Biorad) are plugged with filter paper before filling with 1.1-1.2 ml of resin (AG1-X8 Biorad, 140-14444) suspended in 0.1M formic acid (24 g resin per 50 ml acid). The formic acid is allowed to run out before sealing the syringe tips and filling with 200-300 μL of 0.1M formic acid before storage at 4° C.

On the day of assay, columns are washed with 1 ml of 0.1 M formic acid followed by 1 ml of distilled water. The contents of each assay well are then added to one column and washed with 1 ml distilled water followed by 1 ml of 5 mM sodium tetraborate/60 mM sodium formate. The retained radioactive inositol phosphates are then eluted with 2*1 ml of 1M ammonium formate/0.1M formic acid into 24-well visiplates. Scintillation liquid (1.2 ml UltimaFlow AF) is added to each well and the plate sealed and vortexed before radioactivity is determined by conventional liquid scintillation counting (Microbeta,Perkin Elmer). Unless otherwise stated, all reagents are obtained from Sigma.

Compounds of the instant invention have an IC₅₀ range of 0.0001-100.00 μM (B-IC₅₀).

### CONCLUSIONS

In conclusion, from the foregoing, it is apparent that the present invention provides novel, valuable, and unpredictable applications and uses of the compounds of the present invention, which compounds comprise the active principle according to the present invention, as well as novel pharmaceutical compositions thereof and methods of preparation thereof and of treating therewith, all possessed of the foregoing more specifically-enumerated characteristics and advantages.

The high order of activity of the active agent of the present invention and compositions thereof, as evidenced by the tests reported, is indicative of utility based on its valuable activity in human beings as well as in lower animals. Clinical evaluation in human beings has not been completed, however. It will be clearly understood that the distribution and marketing of any compound or composition falling within the scope of the present invention for use in human beings will of course have to be predicated upon prior approval by governmental agencies, such as the U.S. Federal Food and Drug Administration, which are responsible for and authorized to pass judgment on such questions.

The instant cyclic and acyclic propenone derivatives represent a novel class of Group I mGluR modulators. In view of their potency, they will be useful therapeutics in a wide range of CNS disorders which involve abnormal glutamate neurotransmission.

These compounds accordingly find application in the treatment of the following disorders of a living animal body, especially a human: AIDS-related dementia, Alzheimer's disease, Creutzfeld-Jakob's syndrome, bovine spongiform encephalopathy (BSE) or other prion related infections, diseases involving mitochondrial dysfunction, diseases involving β-amyloid and/or tauopathy such as Down's syndrome, hepatic encephalopathy, Huntington's disease, motor neuron diseases such as amyotrophic lateral sclerosis (ALS), multiple sclerosis (MS), olivoponto-cerebellar atrophy, post-operative cognitive deficit (POCD), Parkinson's disease, Parkinson's dementia, mild cognitive impairment, dementia pugilisitca, vascular and frontal lobe dementia, cognitive impairment, eye injuries or diseases (e.g. glaucoma, retinopathy, macular degeneration), head and spinal cord injuries/trauma, hypoglycaemia, hypoxia (e.g. perinatal), ischaemia (e.g. resulting from cardiac arrest, stroke, bypass operations or transplants), convulsions, glioma and other tumours, inner ear insult (e.g. in tinnitus, sound or drug-induced), L-dopa-induced and tardive dyskinesias.

These compounds also find application in the treatment of the following disorders of a living animal body, especially a human: addiction (nicotine, alcohol, opiate, cocaine, amphetamine, obesity and others), amyotrophic lateral sclerosis (ALS), anxiety and panic disorders, attention deficit hyperactivity disorder (ADHD), restless leg syndrome, hyperactivity in children, autism, convulsions/epilepsy, dementia (e.g. in Alzheimer's disease, Korsakoff syndrome, vascular dementia, HIV infections), major depressive disorder or depression (including that resulting from Borna virus infection) and bipolar manic-depressive disorder, drug tolerance e.g. to opioids, movement disorders, dystonia, dyskinesia (e.g. L-Dopa-induced, tardive dyskinesia or in Huntington's disease), fragile-X syndrome, Huntington's chorea, irritable bowel syndrome (IBS), migraine, multiple sclerosis, muscle spasms, pain (chronic and acute, e.g. inflammatory pain, neuropathic pain, allodynia, hyperalgesia, nociceptive pain), Parkinson's disease, post traumatic stress disorder, schizophrenia (positive and negative symptoms), spasticity, tinnitus, Tourette's syndrome, urinary incontinence and vomiting, pruritic conditions (e.g. pruritis), sleep disorders, micturition disorders, neuromuscular disorder in the lower urinary tract, gastroesophageal reflux disease (GERD), lower esophageal sphincter (LES) disease, functional gastrointestinal disorders, dyspepsia, regurgitation, respiratory tract infection, bulimia nervosa, chronic laryngitis, asthma (e.g. reflux-related asthma), lung disease, eating disorders, obesity and obesity-related disorders.

These compounds also find application in the treatment of indications in of a living animal body, especially a human, wherein a particular condition does not necessarily exist but wherein a particular physiological parameter may be improved through administration of the instant compounds, including cognitive enhancement.

The method-of-treating a living animal body with a compound of the invention, for the inhibition of progression or alleviation of the selected ailment there-in, is as previously stated by any normally-accepted pharmaceutical route, employing the selected dosage which is effective in the alleviation of the particular ailment desired to be alleviated.

Use of the compounds of the present invention in the manufacture of a medicament for the treatment of a living animal for inhibition of progression or alleviation of selected ailments or conditions, particularly ailments or conditions susceptible to treatment with an Group I mGluR modulator, is carried out in the usual manner comprising the step of admixing an effective amount of a compound of the invention with a pharmaceutically-acceptable diluent, excipient, or carrier, and the method-of-treating, pharmaceutical compositions, and use of a compound of the present invention in the manufacture of a medicament.

Representative pharmaceutical compositions prepared by admixing the active ingredient with a suitable pharmaceutically-acceptable excipient, diluent, or carrier, include tablets, capsules, solutions for injection, liquid oral formulations, aerosol formulations, TDS formulations, and nanoparticle formulations, thus to produce medicaments for oral, injectable, or dermal use, also in accord with the foregoing.

The present invention is not to be limited in scope by the specific embodiments described herein. Indeed, various modifications of the invention in addition to those described herein will become apparent to those skilled in the art from the foregoing description. All patents, applications, publications, test methods, literature, and other materials cited herein are hereby incorporated by reference.

We claim:

1. A compound selected from those of Formula I 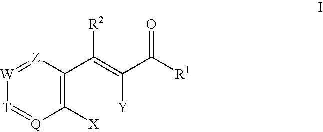
wherein
R¹ represents C₁₋₆alkyl, cycloC₃₋₁₂alkyl, C₂₋₆alkenyl, aryl, arylC₁₋₆alkyl, arylC₂₋₆alkenyl, heteroaryl, heteroarylC₁₋₆-alkyl, arylC₃₋₆cycloalkyl, heteroarylC₂₋₆alkenyl, 2,3-dihydro-1H-indenyl, cycloC₃₋₁₂alkyl or
cycloC₃₋₁₂alkyl-C₁₋₆alkyl, wherein the cycloC₃₋₁₂alkyl is optionally unsaturated and wherein one or more carbon atoms of the cycloC₃₋₁₂alkyl moiety may optionally be replaced by an oxygen atom or an NR⁷-moiety;
R² represents hydrogen or C₁₋₆alkyl;
X represents hydrogen, C₁₋₆alkyl, halogen, cyano, C₁₋₆alkoxy, nitro, or di-(C₁₋₆alkyl)amino;
Y represents hydrogen, halogen, cyano C₁₋₆alkyl, C₁₋₆alkoxy, hydroxyC₁₋₆alkyl, or di-C₁₋₆alkylaminoC₁₋₆alkyl; or
X and Y together may form a bivalent radical selected from OCR⁹R¹⁰, CH₂CR⁹R¹⁰, oxygen, CH₂, and N(R⁸);
Q represents nitrogen or R³—C;
T represents nitrogen or R⁴—C;
W represents nitrogen or R⁵—C;
Z represents nitrogen or R⁶—C;
wherein
R³, R⁴, R⁵ and R⁶ each independently represents a hydrogen atom, a halogen atom, or a group selected from hydroxy, cyano, nitro, C₁₋₆alkyl, hydroxyC₁₋₆alkyl, C₁₋₆alkoxyC₁₋₆alkyl, aryl, arylC₁₋₆alkyl, heteroaryl, C₁₋₆alkoxy, cycloC₃₋₁₂alkoxy, arylC₁₋₆alkoxy, amino, C₁₋₆alkylamino, di-(C₁₋₆alkyl)amino, cycloC₃₋₁₂alkylamino, cycloC₃₋₁₂alkyl-C₁₋₆alkylamino, di-(C₁₋₆alkyl)aminoC₁₋₆alkyl, arylamino, arylC₁₋₆alkylamino, N-aryl-N-C₁₋₆alkylamino, C₁₋₆alkylcarbonylamino, N-C₁₋₆alkyl-N-C₁₋₆alkylcarbonylamino, pyrrolidino, piperidino, 4-C₁₋₆alkyl-piperazino, morpholino, hexamethyleneimino, pyrrolidinylC₁₋₆alkyl, piperidinylC₁₋₆alkyl, morpholinylC₁₋₆alkyl, C₁₋₆alkylsulfonyl, C₁₋₆alkysulfonylamino, C₁₋₆alkylsulfanyl, C₁₋₆alkylaminosulfonyl, and di-(C₁₋₆alkyl)aminosulfonyl;
R⁴ and R⁵ together may form a bivalent radical selected from —(CH₂)₃—, —(CH₂)₄—, —CH═CH—CH═CH—, —(CH₂)₃O—, —OCH₂O—, —O(CH₂)₂O—, and —O(CH₂)₃—;
R⁷ represents hydrogen, C₁₋₆-alkyl, aryl, or cycloC₃₋₁₂alkylC₁₋₆alkyl;
R⁸ represents hydrogen, C₁₋₆alkyl or di-(C₁₋₆-alkyl)aminocarbonyl;
R⁹ and R¹⁰ represent hydrogen or C₁₋₆alkyl;
and optical isomers and pharmaceutically acceptable salts, hydrates, solvates, and polymorphs thereof;
it being understood that:
aryl represents phenyl or naphthyl, or phenyl substituted by one or more substituents, which may be the same or different, selected from halogen, trifluoromethyl, trifluoromethoxy, C₁₋₆alkyl, C₂₋₆alkenyl, C₁₋₆-alkoxy, amino, hydroxy, nitro, cyano, C₁₋₆-alkoxycarbonyl, C₁₋₆alkylamino, di-(C₁₋₆alkyl)amino and C₁₋₆-alkylenedioxy;
heteroaryl represents a (hetero)aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substitued by one or more substituents, which may be the same or different, selected from halogen, trifluoromethyl, C₁₋₆-alkoxy, amino, hydroxy, nitro, cyano, C₁₋₆alkoxycarbonyl, C₁₋₆-alkylamino, and di-(C₁₋₆-alkyl)amino;
if Y represents hydrogen or C₁₋₆alkyl and R¹ represents aryl, then the ring formed by the substituents Q, T, W, and Z may not represent phenyl or substituted phenyl;
if R⁸ represents hydrogen, then R¹ may not represent C₁₋₆-alkyl, phenyl or phenyl substituted by one or more groups selected from halogen, alkoxy, trifluoromethyl, alkyl, nitro, and amino; naphthyl; isoquinolinyl; 2-pyridyl or 2-thienyl;
and the compound of formula I may not represent:
Cyclopropyl(5-methoxy-1H-2-indolyl)-1-methanone,
Cyclobutyl(5-methoxy-1H-2-indolyl)-1-methanone,
1-Adamantan-1-yl-3-quinolin-3-yl-propenone,
(6-Methoxy-2-benzofuran-2-yl)-(3-methoxyphenyl)-methanone,
1-Cyclopropyl-3-(3-methoxypheny)-propenone,
1-(3-Methoxyphenyl)-4,4-dimethyl-pent-1-en-3-one,
1-Adamantan-1-yl-3-(3,4,5-trimethoxyphenyl)-propenone,
1-Adamantan-1-yl-3-phenyl-propenone,
4-(3-oxo-3-(1-adamantyl)-prop-1-enyl)benzonitrile,
1-Adamantan-1-yl-3-(4-nitrophenyl)-propenone,
1-Adamantan-1-yl-3-(4-chlorophenyl)-propenone,
1-Adamantan-1-yl-3-(4-dimethylaminophenyl)-propenone,
1-Adamantan-1-yl-3-(4-isopropylphenyl)-propenone,
1-Adamantan-1-yl-3-(4-methoxyphenyl)-propenone,
1-Adamantan-1-yl-3-(4-fluorophenyl)-propenone,
1-Adamantan-1-yl-3-(2-bromophenyl)-propenone,
1-Adamantan-1-yl-3-(4-benzyloxyphenyl)-propenone,
1-Adamantan-1-yl-3-(4-biphenyl)-propenone,
1-Adamantan-1-yl-3-(4-ethylphenyl)-propenone,
1-Adamantan-1-yl-3-pyridin-2-yl-propenone,
1-Adamantan-1-yl-3-pyridin-3-yl-propenone,
1-Adamantan-1-yl-3-pyridin-4-yl-propenone,
1-Adamantan-1-yl-3-(6-methylpyridin-2-yl)-propenone,
1-Adamantan-1-yl-3-quinolin-4-yl-propenone,
1-Adamantan-1-yl-3-quinolin-2-yl-propenone or
1-Adamantan-1-yl-3-thiophen-2-yl-propenone.

2. A compound of claim 1, wherein R¹ represents C₁₋₆alkyl, cycloC₃₋₁₂alkyl, aryl, arylC₁₋₆alkyl, or arylC₃₋₆cycloalkyl.

3. A compound of claim 2, wherein R¹ represents t-butyl, cyclopropyl, adamantyl, optionally substituted phenyl, optionally substituted benzyl, α,α-dimethylbenzyl or optionally substituted 1-phenyl-cyclopent-1-yl.

4. A compound of claim 1, wherein R² represents hydrogen or C₁₋₆alkyl.

5. A compound of claim 4, wherein R² represents hydrogen or methyl.

6. A compound of claim 1, wherein R³, R⁴, R⁵ and R⁶, which may be the same or different, represent hydrogen, halogen, hydroxy, cyano, nitro, C₁₋₆alkyl which may be optionally substituted by C₁₋₆alkoxy, hydroxyC₁₋₆alkyl, heteroaryl, C₁₋₆alkoxy, arylC₁₋₆alkoxy, amino, C₁₋₆alkylamino, di-(C₁₋₆alkyl)amino, C₁₋₆alkylcarbonylamino, N-C₁₋₆alkylN-C₁₋₆alkylcarbonylamino, pyrrolidino, piperidino, 4-C₁₋₆alkyl-piperazino or morpholino.

7. A compound of claim 6, wherein R³, R⁴, R⁵ and R⁶, which may be the same or different, represent hydrogen, fluoro, bromo, chloro, hydroxy, cyano, nitro, methyl, methoxymethyl, hydroxymethyl, pyridyl, oxazolyl, thiazolyl, methoxy, ethoxy, benzyloxy, amino, dimethylamino, pyrrolidino, piperidino, morpholino, 4-methyl-piperazino, acetylamino or N-methyl-N-acetylamino.

8. A compound of claim 1, wherein R⁴ and R⁵ together form a bivalent radical selected from —CH═CH—CH═CH— and —O(CH₂)₂O—.

9. A compound of claim 1, wherein one of Q, T, W and Z represents a nitrogen atom and the remaining of Q, T, W and Z represent optionally substituted carbon atoms.

10. A compound of claim 9, wherein R¹ represents C₁₋₆alkyl, cycloC₃₋₁₂alkyl, aryl, arylC₁₋₆alkyl, or arylC₃₋₆cycloalkyl.

11. A compound of claim 9, wherein R² represents hydrogen or C₁₋₆alkyl.

12. A compound of claim 9, wherein R³, R⁴, R⁵ and R⁶, which may be the same or different, represent hydrogen, halogen, hydroxy, cyano, nitro, C₁₋₆alkyl which may be optionally substituted by C₁₋₆alkoxy, hydroxyC₁₋₆alkyl, heteroaryl, C₁₋₆alkoxy, arylC₁₋₆alkoxy, amino, C₁₋₆alkylamino, di-(C₁₋₆alkyl)amino, C₁₋₆alkylcarbonylamino, N-C₁₋₆alkylN-C₁₋₆alkylcarbonylamino, pyrrolidino, piperidino, 4-C₁₋₆alkyl-piperazino or morpholino.

13. A compound of claim 1, which is selected from those of Formula IA 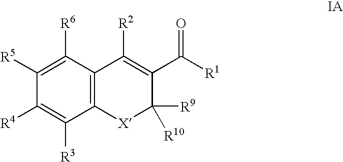
wherein X′ represents oxygen or CH₂;
and optical isomers and pharmaceutically acceptable salts, hydrates, solvates, and polymorphs thereof.

14. A compound of claim 13, wherein R¹ represents C₁₋₆alkyl, cycloC₃₋₁₂alkyl, aryl, arylC₁₋₆alkyl, or arylC₃₋₆cycloalkyl.

15. A compound of claim 14, wherein R¹ represents adamantyl or phenyl.

16. A compound of claim 13, wherein R³, R⁴, R⁵ and R⁶, which may be the same or different, represent hydrogen, halogen, C₁₋₆alkyl, heteroaryl, C₁₋₆alkoxy, arylC₁₋₆alkoxy, amino, C₁₋₆alkylamino, di-(C₁₋₆alkyl)amino, C₁₋₆alkylcarbonylamino, pyrrrolidino, 4-C₁₋₆alkyl-piperazino or morpholino.

17. A compound of claim 16, wherein R³ represents hydrogen or bromo.

18. A compound of claim 16, wherein R⁴ represents hydrogen, bromo, oxazolyl, thiazolyl, methoxy, dimethylamino, acetylamino, pyrrolidino, piperidino, morpholino or 4-methyl-piperazino.

19. A compound of claim 1, which is selected from those of Formula IB 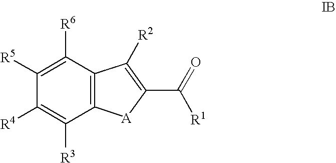
wherein A represents oxygen, CH₂, or NR⁸;
and optical isomers and pharmaceutically acceptable salts, hydrates, solvates, and polymorphs thereof.

20. A compound of claim 19, wherein R⁸ represents hydrogen, methyl or diethylaminocarbonyl.

21. A compound of claim 19, wherein R¹ represents C₁₋₆alkyl, cycloC₃₋₁₂alkyl, aryl, arylC₁₋₆alkyl, or arylC₃₋₆cycloalkyl.

22. A compound of claim 21, wherein R¹ represents t-butyl, adamantyl, phenyl substituted by one or more methoxy groups, phenyl substituted by one or more methyl groups, phenyl substituted by a group selected from nitro, cyano, fluoro and trifluoromethoxy, α,α-dimethylbenzyl, which may be optionally substituted on the phenyl ring by chloro, or 1-(4-chlorophenyl)-cyclopent-1-yl.

23. A compound of claim 19, wherein R² represents hydrogen or C₁₋₆alkyl.

24. A compound of claim 23, wherein R² represents hydrogen or methyl.

25. A compound of claim 19, wherein R³, R⁴, R⁵ and R⁶, which may be the same or different, represent hydrogen, halogen, hydroxy, cyano, nitro, C₁₋₆alkyl, hydroxyC₁₋₆alkyl, heteroaryl, C₁₋₆alkoxy, arylC₁₋₁₆alkoxy, amino, C₁₋₆alkylamino, di-(C₁₋₆alkyl)amino, C₁₋₆alkylcarbonylamino, N—C₁₋₆alkylN-C₁₋₆alkylcarbonylamino, pyrrolidino, piperidino, 4-C₁₋₆alkyl-piperazino or morpholino.

26. A compound of claim 25, wherein R³ represents hydrogen or ethoxy.

27. A compound of claim 25, wherein R⁴ represents hydrogen, fluoro, bromo, methoxy, amino, diethylamino, pyrrolidino, piperidino, morpholino or acetylamino.

28. A compound of claim 25, wherein R⁵ represents hydrogen, bromo, hydroxy, cyano, nitro, methoxy, benzyloxy, acetylamino or N-methyl-N-acetylamino.

29. A compound of claim 26, wherein R⁶ represents hydrogen, hydroxymethyl or methoxy.

30. A compound of claim 1, which is selected from those of Formula IC 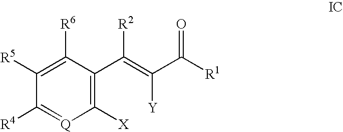
and optical isomers and pharmaceutically acceptable salts, hydrates, solvates, and polymorphs thereof.

31. A compound of claim 30, wherein R¹ represents C₁₋₆alkyl, cycloC₃₋₁₂alkyl, aryl, arylC₁₋₆alkyl, or arylC₃₋₆cycloalkyl.

32. A compound of claim 31, wherein R¹ represents t-butyl, cyclopropyl or adamantyl.

33. A compound of claim 30, wherein R² represents hydrogen or C₁₋₆alkyl.

34. A compound of claim 30, wherein X represents hydrogen, C₁₋₆alkyl or C₁₋₆-alkoxy.

35. A compound of claim 34, wherein X represents hydrogen or methoxy.

36. A compound of claim 30, wherein Y represents hydrogen or C₁₋₆alkyl.

37. A compound of claim 30, wherein R³, R⁴, R⁵ and R⁶, which may be the same or different, represent hydrogen, halogen, hydroxy, cyano, nitro, C₁₋₆alkyl which may be optionally substituted by C₁₋₆alkoxy, hydroxyC₁₋₆alkyl, heteroaryl, C₁₋₆alkoxy, arylC₁₋₆alkoxy, amino, C₁₋₆alkylamino, di-(C₁₋₆alkyl)amino, C₁₋₆alkylcarbonylamino, N-C₁₋₆alkylN-C₁₋₆alkylcarbonylamino, pyrrolidino, piperidino, 4-C₁₋₆alkyl-piperazino or morpholino.

38. A compound of claim 37, wherein R³ represents hydrogen or methoxy.

39. A compound of claim 37, wherein R⁴ represents hydrogen or methoxy.

40. A compound of claim 37, wherein R⁵ represents hydrogen, methyl, methoxy or benzyloxy.

41. A compound of claim 30, wherein R⁴ and R⁵ together form a bivalent radical selected from —CH═CH—CH═CH— and —O(CH₂)₂O—.

42. A compound of claim 1, which is selected from:
1-Cyclopropyl-3-(3-methoxyphenyl)-propenone,
1-Adamantan-1-yl-3-(3-methoxyphenyl)-propenone,
1-Cyclopropyl-3-(3,5-dimethoxy-phenyl)-propenone,
1-Adamantan-1-yl-3-(3,5-dimethoxy-phenyl)-propenone,
1-Cyclopropyl-3-quinolin-3-yl-propenone,
4,4-Dimethyl-1-quinolin-3-yl-pent-1-en-3-one,
1-(3,5-Dimethoxy-phenyl)-4,4-dimethyl-pent-1-en-3-one,
1-Adamantan-1-yl-3-(2,5-dimethoxy-phenyl)-propenone,
1-Adamantan-1-yl-3-(4-methoxy-3-methyl-phenyl)-propenone,
1-Adamantan-1-yl-3-(2,3-dihydrobenzo[1,4]dioxin-6-yl)-propenone,
2-(Adamantane-1-carbonyl)-3-(2,3-dihydro-benzo[1,4]dioxin-6-yl)-acrylonitrile,
1-Adamantan-1-yl-3-(3-benzyloxy-phenyl)-propenone,
1-Adamantan-1-yl-3-(3,4,5-trimethoxy-phenyl)-propenone and
1-(3-Methoxy-phenyl)-4,4-dimethyl-pent-1-en-3-one.

43. A compound of claim 1, which is selected from:
Adamantan-1-yl-(2H-chromen-3-yl)-methanone,
(6-Bromo-2H-chromen-3-yl)-phenylmethanone and
Adamantan-1-yl-(7-methoxy-2H-chromen-3-yl)-methanone.

44. A compound of claim 1, which is selected from:
Adamantan-1-yl-benzofuran-2-yl-methanone,
Adamantan-1-yl-(7-ethoxy-benzofuran-2-yl)-methanone,
Adamantan-1-yl-(5-methoxy-benzofuran-2-yl)-methanone,
Benzofuran-2-yl-(2,5-dimethoxy-phenyl)-methanone,
(2,5-Dimethoxy-phenyl)-(5-methoxy-benzofuran-2-yl)-methanone,
(2,5-Dimethoxy-phenyl)-(6-methoxy-benzofuran-2-yl)-methanone,
(2,5-Dimethoxy-phenyl)-(7-ethoxy-benzofuran-2-yl)-methanone,
Adamantan-1-yl-(6-diethylamino-benzofuran-2-yl)-methanone,
(6-Diethylamino-benzofuran-2-yl)-(3-methoxy-phenyl)-methanone,
(6-Diethylamino-benzofuran-2-yl)-(2,5-dimethoxy-phenyl)-methanone,
(6-Methoxy-benzofuran-2-yl)-(3-methoxy-phenyl)-methanone,
(5,4-Dimethyl-phenyl)-(6-methoxy-benzofuran-2-yl)-methanone,
Adamantan-1-yl-(5-bromo-benzofuran-2-yl)-methanone,
Benzofuran-2-yl-(2,5-dimethoxy-phenyl)-methanone,
Benzofuran-2-yl-(2,3-dihydrobenzo[1,4]dioxin-6-yl)-methanone,
1-(6-Methoxy-benzofuran-2-yl)-2-methyl-2-phenyl-propan-1-one,
Adamantan-1-yl-(5-nitro-benzofuran-2-yl)-methanone,
Adamantan-1-yl-(4-methoxy-benzofuran-2-yl)-methanone,
Adamantan-1-yl-(4-hydroxymethyl-7-methyl-furo[2,3-c]pyridin-2-yl)-methanone,
Adamantan-1-yl-(6-methoxy-3-methyl-benzofuran-2-yl)-methanone,
(6-Diethylamino-benzofuran-2-yl)-(2-nitro-phenyl)-methanone,
Adamantan-1-yl-(6-fluoro-3-methyl-benzofuran-2-yl)-methanone,
(6-Diethylamino-benzofuran-2-yl)-(2,3-dihydro-benzo[1,4]dioxin-6-yl)-methanone,
(6-Diethylamino-benzofuran-2-yl)-p-tolyl methanone,
4-(6-Diethylamino-benzofuran-2-carbonyl)-benzonitrile,
(6-Diethylamino-benzofuran-2-yl)-(2,4-dimethyl-phenyl)-methanone,
Adamantan-1-yl-(6-methoxy-benzofuran-2-yl)-methanone,
Adamantan-1-yl-(6-bromo-benzofuran-2-yl)-methanone,
Adamantan-1-yl-(6-morpholin-4-yl-benzofuran-2-yl)-methanone,
Adamantan-1-yl-(6-piperidin-1-yl-benzofuran-2-yl)-methanone,
Adamantan-1-yl-(6-pyrrolidin-1-yl-benzofuran-2-yl)-methanone,
Adamantan-1-yl-(6-pyridin-3-yl-benzofuran-2-yl)-methanone,
Adamantan-1-yl-(6-amino-benzofuran-2-yl)-methanone,
N-[2-(Adamantane-1-carbonyl)-benzofuran-6-yl]-acetamide,
Adamantan-1-yl-furo[3,2-c]pyridin-2-yl-methanone,
Adamantan-1-yl-(4,7-dimethyl-furo[2,3-c]pyridin-2-yl)-methanone and
Adamantan-1-yl-(4-methoxymethyl-7-methyl-furo[2,3-c]pyridin-2-yl)-methanone.

45. A compound of claim 1, which is selected from:
2-[2-(4-Chloro-phenyl)-2-methyl-propionyl]-5-methoxy-indole-1-carboxylic acid diethylamide,
2-(4-Chloro-phenyl)-1-(5-methoxy-1H-indol-2-yl)-2-methyl-propan-1-one,
(5-Bromo-1-methyl-1H-indol-2-yl)-(4-fluoro-phenyl)-methanone,
N-[2-(4-Fluoro-benzoyl)-1-methyl-1H-indol-5-yl]-acetamide,
Adamantan-1-yl-(5-hydroxy-1H-indol-2-yl)-methanone,
Adamantan-1-yl-(5-benzyloxy-1H-indol-2-yl)-methanone,
(5-Benzyloxy-1H-indol-2-yl)-[1-(4-chloro-phenyl)-cyclopentyl]-methanone,
2-(Adamantane-1-carbonyl)-1H-indole-5-carbonitrile,
Adamantan-1-yl-(5-methoxy-1H-indol-2-yl)-methanone,
[1-(4-Chloro-phenyl)-cyclopentyl]-(5-methoxy-1H-indol-2-yl)-methanone,
(5-Bromo-1-methyl-1H-indol-2-yl)-p-tolyl-methanone,
(5-Benzyloxy-1-methyl-1H-indol-2-yl)-(4-fluoro-phenyl)-methanone,
(5-Benzyloxy-1-methyl-1H-indol-2-yl)-p-tolyl-methanone,
N-[1-Methyl-2-(4-methyl-benzoyl)-1H-indol-5-yl]-acetamide,
(5-Methoxy-1-methyl-1H-indol-2-yl)-p-tolyl-methanone,
1-(5-Benzyloxy-1-methyl-1H-indol-2-yl)-2,2-dimethyl-propan-1-one,
[1-(4-Chloro-phenyl)-cyclopentyl]-(6-fluoro-1H-indol-2-yl)-methanone,
2-(4-Chloro-phenyl)-1-(6-fluoro-1H-indol-2-yl)-2-methyl-propan-1-one,
Adamantan-1-yl-(6-fluoro-1H-indol-2-yl)-methanone,
N-[2-(4-Fluoro-benzoyl)-1-methyl-1H-indol-5-yl]-N-methyl-acetamide,
N-Methyl-N-[1-methyl-2-(4-methyl-benzoyl)-1H-indol-5-yl]-acetamide,
Adamantan-1-yl-(5-fluoro-1H-indol-2-yl)-methanone and
1-(5-Hydroxy-1-methyl-1H-indol-2-yl)-2,2-dimethyl-propan-1-one.

46. A compound of claim 1, which is selected from:
Adamantan-1-yl-(1H-inden-2-yl)-methanone and
(1H-Inden-2-yl)-(4-trifluoromethoxy-phenyl)-methanone.

47. A compound of claim 1, which is selected from:
Adamantan-1-yl-(2H-pyrano[3,2-c]pyridin-3-yl)-methanone,
Adamantan-1-yl-(7-bromo-2H-chromen-3-yl)-methanone,
N-[3-(Adamantane-1-carbonyl)-2H-chromen-7-yl]-acetamide,
Adamantan-1-yl-(7-dimethylamino-2H-chromen-3-yl)-methanone,
Adamantan-1-yl-(7-pyrrolidin-1-yl-2H-chromen-3-yl)-methanone,
Adamantan-1-yl-(7-piperidin-2H-chromen-3-yl)-methanone,
Adamantan-1-yl-(7-morpholin-4-yl-2H-chromen-3-yl)-methanone,
Adamantan-1-yl-[7-(4-methyl-piperazin-1-yl-2H-chromen-3-yl]-methanone,
Adamantan-1-yl-(7-oxazol-2-yl-2H-chromen-3-yl]-methanone and
Adamantan-1-yl-(7-thiazol-2-yl-2H-chromen-3-yl]-methanone.

48. A method of treating a living animal body, including a human, afflicted with a condition associated with abnormal glutamate neurotransmission or in which modulation of Group I mGluR receptors results in therapeutic benefit, comprising the step of administering to the living animal body, including a human, an amount of a compound of Formula I 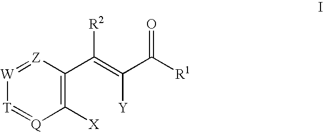
wherein
R¹ represents C₁₋₆alkyl, cycloC₃₋₁₂alkyl, C₂₋₆alkenyl, aryl, arylC₁₋₆alkyl, arylC₂₋₆alkenyl, heteroaryl, heteroarylC₁₋₆alkyl, aryl-C₃₋₆cycloalkyl, heteroarylC₂₋₆alkenyl, 2,3-dihydro-1H-indenyl, cycloC₃₋₁₂alkyl or cycloC₃₋₁₂alkyl-C₁₋₆alkyl, wherein the cycloC₃₋₁₂alkyl is optionally unsaturated and wherein one or more carbon atoms of the cycloC₃₋₁₂alkyl moiety may optionally be replaced by an oxygen atom or an NR⁷-moiety;
R² represents hydrogen or C₁₋₆-alkyl;
X represents hydrogen, C₁₋₆-alkyl, halogen, cyano, C₁₋₆alkoxy, nitro, or di-(C₁₋₆alkyl)amino;
Y represents hydrogen, halogen, cyano, C₁₋₆alkyl, C₁₋₆alkoxy, hydroxyC₁₋₆-alkyl, or di-C₁₋₆alkylaminoC₁₋₆alkyl; or
X and Y together may form a bivalent radical selected from OCR⁹R¹⁰, CH₂CR⁹R¹⁰, oxygen, CH₂, and N(R⁸);
Q represents nitrogen or R³—C;
T represents nitrogen or R⁴—C;
W represents nitrogen or R⁵—C;
Z represents nitrogen or R⁶—C;
wherein
R³, R⁴, R⁵ and R⁶ each independently represents a hydrogen atom, a halogen atom, or a group selected from hydroxy, cyano, nitro, C₁₋₆alkyl, hydroxyC₁₋₆alkyl, C₁₋₆alkoxyC₁₋₆alkyl, aryl, arylC₁₋₆alkyl, heteroaryl, C₁₋₆alkoxy, cycloC₃₋₁₂alkoxy, arylC₁₋₆alkoxy, amino, C₁₋₆-alkylamino, di-(C₁₋₆alkyl)amino, cycloC₃₋₁₂alkylamino, cycloC₃₋₁₂alkyl-C₁₋₆alkylamino, di-(C₁₋₆alkyl)aminoC₁₋₆alkyl, arylamino, arylC₁₋₆alkylamino, N-aryl-N-C₁₋₆alkylamino, C₁₋₆alkylcarbonylamino, N-C₁₋₆alkyl-N-C₁₋₆-alkylcarbonylamino, pyrrolidino, piperidino, 4-C₁₋₆alkyl-piperazino, morpholino, hexamethyleneimino, pyrrolidinylC₁₋₆alkyl, piperidinylC₁₋₆alkyl, morpholinylC₁₋₆alkyl, C₁₋₆alkylsulfonyl, C₁₋₆alkylsulfonylamino, C₁₋₆alkylsulfanyl, C₁₋₆alkylaminosulfonyl, and di-(C₁₋₆alkyl)aminosulfonyl;
R⁴ and R⁵ together may form a bivalent radical selected from —(CH₂)₃—, —(CH₂)₄—, —CH═CH—CH═CH—, —(CH₂)₃O—, —OCH₂O—, —O(CH₂)₂O—, and —O(CH₂)₃—;
R⁷ represents hydrogen, C₁₋₆alkyl, aryl, or cycloC₃₋₁₂alkyl-C₁₋₆alkyl;
R⁸ represents hydrogen, C₁₋₆alkyl or di-(C₁₋₆-alkyl)aminocarbonyl;
R⁹ and R¹⁰ represent hydrogen or C₁₋₆alkyl;
it being understood that:
aryl represents phenyl or naphthyl, or phenyl substituted by one or more substituents, which may be the same or different, selected from halogen, trifluoromethyl, trifluoromethoxy, C₁₋₆-alkyl, C₂₋₆alkenyl, C₁₋₆-alkoxy, amino, hydroxy, nitro, cyano, C₁₋₆-alkoxycarbonyl, C₁₋₆alkylamino, di-(C₁₋₆alkyl)amino and C₁₋₆alkylenedioxy;
heteroaryl represents a (hetero)aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substitued by one or more substituents, which may be the same or different, selected from halogen, trifluoromethyl, C₁₋₆-alkoxy, amino, hydroxy, nitro, cyano, C₁₋₆alkoxycarbonyl, C₁₋₆-alkylamino, and di-(C₁₋₆-alkyl)amino;
its optical isomers and pharmaceutically acceptable acid and base addition salts thereof;
which is effective for alleviation of the condition.

49. The method of claim 48, wherein the condition associated with abnormal glutamate neurotransmission or in which modulation of Group I mGluR receptors results in therapeutic benefit is selected from: AIDS-related dementia, Alzheimer's disease, Creutzfeld-Jakob's syndrome, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), multiple sclerosis (MS), olivoponto-cerebellar atrophy, post-operative cognitive deficit (POCD), Parkinson's disease, Parkinson's dementia, mild cognitive impairment, dementia pugilisitca, vascular and frontal lobe dementia, cognitive impairment, eye injuries, eye disorders, glaucoma, retinopathy, macular degeneration, head and spinal cord injuries, trauma, hypoglycaemia, hypoxia, perinatal hypoxia, ischaemia resulting from cardiac arrest, stroke, bypass operations or transplants, convulsions, glioma and other tumours, inner ear insult, tinnitus, sound or drug-induced tinnutis, L-dopa-induced dyskinesias, tardive dyskinesias, addiction, nicotine addiction, alcohol addiction, opiate addiction, cocaine addiction, amphetamine addiction, anxiety and panic disorders, attention deficit hyperactivity disorder (ADHD), restless leg syndrome, hyperactivity in children, autism, convulsions, epilepsy, dementia, Alzheimer's disease, Korsakoff syndrome, vascular dementia, dementia related to HIV infections, major depressive disorder, depression resulting from Borna virus infection, bipolar manic-depressive disorder, drug tolerance, drug tolerance to opioids, movement disorders, dystonia, dyskinesias, L-Dopa-induced dyskinesias, tardive dyskinesias, Huntington's disease, fragile-X syndrome, Huntington's chorea, irritable bowel syndrome (IBS), migraine, multiple sclerosis, muscle spasms, chronic pain, acute pain, inflammatory pain, neuropathic pain, allodynia, hyperalgesia, nociceptive pain, Parkinson's disease, post traumatic stress disorder, schizophrenia, spasticity, Tourette's syndrome, urinary incontinence and vomiting, pruritic conditions, pruritis, sleep disorders, micturition disorders, neuromuscular disorder in the lower urinary tract, gastroesophageal reflux disease (GERD), lower esophageal sphincter (LES) disease, functional gastrointestinal disorders, dyspepsia, regurgitation, respiratory tract infection, bulimia nervosa, chronic laryngitis, asthma, reflux-related asthma, lung disease, eating disorders, obesity and obesity-related disorders, agoraphobia, generalized anxiety disorder, obsessive-compulsive disorder, panic disorder, posttraumatic stress disorder, social phobia, substance-induced anxiety disorder, delusional disorder, schizoaffective disorder, schizophreniform disorder, substance-induced psychotic disorder, and delirium.

50. The method of claim 48, wherein the condition associated with abnormal glutamate neurotransmission or in which modulation of Group I mGluR receptors results in therapeutic benefit is selected from: addiction, neuropathic pain, L-dopa-induced and tardive dyskinesias, ALS, fragile-X syndrome, Parkinson's disease, anxiety disorders, epilepsy, positive and/or negative symptoms of schizophrenia, and cognitive impairment.

51. A method of treating a living animal body, including a human, for a condition in which a particular physiological parameter is improved through administration of a Group I mGluR modulator, comprising the step of administering to the living animal body, including a human, an amount of a compound of Formula I 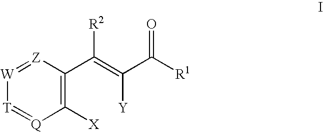
wherein
R¹ represents C₁₋₆alkyl, cycloC₃₋₁₂alkyl, C₂₋₆alkenyl, aryl, arylC₁₋₆alkyl, arylC₂₋₆alkenyl, heteroaryl, heteroarylC₁₋₆alkyl, aryl-C₃₋₆cycloalkyl, heteroarylC₂₋₆alkenyl, 2,3-dihydro-1H-indenyl, cycloC₃₋₁₂alkyl or cycloC₃₋₂alkyl-C₁₋₆alkyl, wherein the cycloC₃₋₁₂alkyl is optionally unsaturated and wherein one or more carbon atoms of the cycloC₃₋₁₂alkyl moiety may optionally be replaced by an oxygen atom or an NR⁷-moiety;
R² represents hydrogen or C₁₋₆alkyl;
X represents hydrogen, C₁₋₆alkyl, halogen, cyano, C₁₋₆alkoxy, nitro, or di-(C₁₋₆alkyl)amino;
Y represents hydrogen, halogen, cyano, C₁₋₆alkyl, C₁₋₆alkoxy, hydroxyC₁₋₆alkyl, or di-C₁₋₆-alkylaminoC₁₋₆alkyl; or
X and Y together may form a bivalent radical selected from OCR⁹R¹⁰, CH₂CR⁹R¹⁰, and CH₂CH₂; or
X and Y together may form a bivalent radical selected from oxygen, CH₂, and N(R⁸);
Q represents nitrogen or R³—C;
T represents nitrogen or R⁴—C;
W represents nitrogen or R⁵—C;
Z represents nitrogen or R⁶—C;
wherein
R³, R⁴, R⁵ and R⁶ each independently represents a hydrogen atom, a halogen atom, or a group selected from hydroxy, cyano, nitro, C₁₋₆alkyl, hydroxyC₁₋₆alkyl, C₁₋₆alkoxyC₁₋₆alkyl, aryl, arylC₁₋₆alkyl, heteroaryl, C₁₋₆alkoxy, cycloC₃₋₁₂alkoxy, arylC₁₋₆alkoxy, amino, C₁₋₆alkylamino, di-(C₁₋₆alkyl)amino, cycloC₃₋₁₂alkylamino, cycloC₃₋₁₂alkyl-C₁₋₆alkylamino, di-(C₁₋₆alkyl)aminoC₁₋₆alkyl, arylamino, arylC₁₋₆alkylamino, N-aryl-N-C₁₋₆alkylamino, C₁₋₆alkylcarbonylamino, N-C₁₋₆alkyl-N-C₁₋₆alkylcarbonylamino, pyrrolidino, piperidino, 4-C₁₋₆alkyl-piperazino, morpholino, hexamethyleneimino, pyrrolidinylC₁₋₆alkyl, piperidinylC₁₋₆alkyl, morpholinylC₁₋₆alkyl, C₁₋₆alkylsulfonyl, C₁₋₆alkylsulfonylamino, C₁₋₆alkylsulfanyl, C₁₋₆alkylaminosulfonyl, and di-(C₁₋₆alkyl)aminosulfonyl;
R⁴ and R⁵ together may form a bivalent radical selected from —(CH₂)₃—, —(CH₂)₄—, —CH═CH—CH═CH—, —(CH₂)₃O—, —OCH₂O—, —O(CH₂)₂O—, and —O(CH₂)₃—;
R⁷ represents hydrogen, C₁₋₆-alkyl; aryl, or cycloC₃₋₁₂alkyl-C₁₋₆alkyl;
R⁸ represents hydrogen, C₁₋₆-alkyl or di-(C₁₋₆-alkyl)aminocarbonyl;
R⁹ and R¹⁰ represent hydrogen or C₁₋₆-alkyl;
it being understood that:
aryl represents phenyl or naphthyl, or phenyl substituted by one or more substituents, which may be the same or different, selected from halogen, trifluoromethyl, trifluoromethoxy, C₁₋₆-alkyl, C₂₋₆alkenyl, C₁₋₆-alkoxy, amino, hydroxy, nitro, cyano, C₁₋₆-alkoxycarbonyl, C₁₋₆alkylamino, di-(C₁₋₆-alkyl)amino and C₁₋₆alkylenedioxy;
heteroaryl represents a (hetero)aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substitued by one or more substituents, which may be the same or different, selected from halogen, trifluoromethyl, C₁₋₆alkoxy, amino, hydroxy, nitro, cyano, C₁₋₆-alkoxycarbonyl, C₁₋₆-alkylamino, and di-(C₁₋₆-alkyl)amino;
and optical isomers, pharmaceutically acceptable salts, hydrates, solvates, and polymorphs thereof;
which is effective for improvement of the physiological parameter.

52. The method of claim 51, wherein the condition is selected from cognitive enhancement and neuroprotection.

53. A pharmaceutical composition comprising as active ingredient a compound of claim 1 together with one or more pharmaceutically acceptable excipients or vehicles.
